SEQUENCE LISTING (2 sequences)

SEQ ID NO: 1 - protein, 9 aa, Artificial Sequence
  Synthetic construct
RKKRRQRRR

SEQ ID NO: 2 - DNA, 27 bp, Artificial Sequence
  Synthetic construct
aggaagaagcggagacagcgacgaaga

(19) United States
(12) Patent Application Publication
(10) Pub. No.: US 2014/0109246 A1
(43) Pub. Date: Apr. 17, 2014

(54) METHODS OF GENERATING XENOCHIMAERIC MICE WITH TUMOR AND HEMATOPOIETIC SYSTEM FROM THE SAME HETEROLOGOUS SPECIES

(71) Applicant: The Regents of the University of Colorado, a body corporate, Denver, CO (US)

(72) Inventors: Antonio JIMENO, Englewood, CO (US); Yosef REFAELI, Denver, CO (US); Xiao-Jing WANG, Greenwood Village, CO (US); Dennis ROOP, Greenwood Village, CO (US)

(73) Assignee: The Regents of the University of Colorado, a body corporate, Denver, CO (US)

(21) Appl. No.: 14/035,780

(22) Filed: Sep. 24, 2013

Related U.S. Application Data
(60) Provisional application No. 61/705,050, filed on Sep. 24, 2012.

(51) Int. Cl. A61K 49/00

(52) U.S. Cl. CPC A61K 49/0008
USPC 800/10; 424/9.2; 435/7.23; 514/19.3; 514/44 R; 424/174.1; 424/178.1; 424/277.1

(57) ABSTRACT

The present invention provides methods for the generation of xenochimaeric animals; for example, xenochimaeric mice, comprising bone marrow progenitor cells and tumors from a heterologous animal. In some aspects, the invention provides xenochimaeric mice bearing humanized bone marrow and human tumors. Such animals are a model system for the study of human tumor stroma, for drug discovery, and for the treatment of human cancers.

### CROSS-REFERENCE TO RELATED APPLICATIONS

This application claims the benefit of priority under 35 USC §119 to provisional application No. 61/705,050, filed Sep. 24, 2012, the entire content of which is incorporated herein by reference.

### FUNDING STATEMENT

This invention was made with government support under grant number W81XWN-10-1-0798 awarded by Army/Medical Research Materiel and Command, National Institutes of Health Cancer Center Support Grant P30 CA046934, R21DE019712, R01 CA149456, R01 CA117802-06, and P30 AR057212-02. The government has certain rights in the invention.

### FIELD OF INVENTION

The present invention provides methods for generating xenochimaeric mice with tumors and hematopoietic system from the same heterologous species.

### BACKGROUND OF THE INVENTION

Conventional drug development typically begins with cancer cell line-based in vitro screens followed by limited in vivo testing in cell line-derived tumors (Boyd, M. (1997) in Anticancer drug development guide:preclinical screening, clinical trials, and approval. T. B, ed. Totowa: Humana Press. 23:1985-1992; Johnson, J I et al., (2001) Br J Cancer 84:1424-1431). However, this approach poorly predicts clinical efficacy because cell lines become homogeneous and are no longer dependent on epithelial-stromal interactions critical for in vivo oncogenesis (Engelholm, S A et al., (1985) Eur J Cancer Clin Oncol 21:815-824; Hausser, H J and Brenner, R E (2005) Biochem Biophys Res Commun 333:216-222; De Wever, O and Mareel, M (2003) J Pathol 200:429-447). In patient explant models, patient tumors are directly implanted into immune-deficient mice. Direct explants preserve key features that cells in culture derived from the same explant irreversibly lose (Daniel, V C, et al., (2009) Cancer Res 69:3364-3373).

An example of a mouse model comprising a human immune hematopoietic progenitor cells and human tumor cells is described in United States Patent Application Publication No. 2007/0118914. Methods of conditionally immortalizing long term stem cells are described in United States Patent Application Publication No. 2010/0297763.

What is needed is a non-human animal model for studying a heterologous system (human or other non-human animal) in which the non-human animal bears a heterologous bone marrow and tumor so heterologous stroma home into the heterologous tumor, thereby recreating the original tumor-non-tumor interface.

All references cited herein, including patent applications and publications, are incorporated by reference in their entirety.

### BRIEF SUMMARY OF THE INVENTION

The invention relates to xenochimaeric non-human animal hosts (e.g. mice, guinea pigs, rabbits, and pigs) comprising hematopoietic cells from a heterologous animal (e.g. human or other non-human animal). The particular non-human animal hosts may be selected to provide advantages regarding similarities of systems including, but not limited to, the immune and endothelial system. These xenochimaeric non-human animal hosts are paired with tumors from heterologous species (e.g. tumors that are syngeneic or autologous to the hematopoietic cells in the non-human animal hosts) to function as animal models for cancer, where the tumor stroma is populated with cells of the heterologous species, rather than only stroma from the non-human host animal, thus more closely mimicking the tumor in its native environment. In some embodiments, the heterologous species is a human. In some embodiments, the heterologous species is a non-human animal (e.g. a domesticated animal, a wild animal, a human companion animal, a zoo animal, a farm animal). In some embodiments, the non-human animal is a canine (e.g. dog) or a feline (e.g. cat). For example, canine HSCs and tumors may be engrafted on immunosuppressed mice (or other non-human animal hosts) to create a model for assessing targeted and immune therapies for melanoma, lymphoma and osteosarcoma, for example. In some embodiments, the HSCs are introduced to the mice about 8 to about 10 weeks prior to introduction of the tumor to the mice. In some embodiments, the introduction of the HSCs and the tumor result in formation of stroma corresponding to the heterologous animal. In some embodiments, the introduction of the HSCs and the tumor result in reversion in one or more of tumor phenotype or tumor genotoype towards the phenotype or genotype of the tumor initially isolated from the heterologous animal.

The invention provides methods for producing a xenochimaeric non-human animal host (e.g. mouse, guinea pigs, rabbits, and pigs), the method comprising a) introducing heterologous hematopoietic stem cells (HSCs) from a heterologous animal to the non-human animal (e.g. mouse, guinea pigs, rabbits, and pigs), and b) introducing a malignant or benign tumors from the heterologous animal to the non-human animal (e.g. mouse, guinea pigs, rabbits, and pigs). In some embodiments, the heterologous animal is a non-human animal (e.g. a domesticated animal, a wild animal, a human companion animal, a zoo animal, a farm animal. In some embodiments, the non-human animal is a canine (e.g. dog) or a feline (e.g. cat). In some embodiments, the heterologous animal is a human. In some embodiments, the HSCs and the tumor are from the same individual. In some embodiments, the individual is a cancer patient.

In some embodiments, the invention provides methods for producing a xenochimaeric non-human animal host (e.g. mouse, guinea pigs, rabbits, and pigs) wherein the non-human animal (e.g. mouse, guinea pigs, rabbits, and pigs) is an immunodeficient. In some embodiments, the immunodeficient non-human animal host (e.g. mouse, guinea pigs, rabbits, and pigs) lacks one or more of T cells, NKT cells, B cells and NK cells. In some embodiments, the immunodeficient mouse is a nu⁻/nu⁻ mouse, an NSG (NOD/SCID/gc^{-/-}) mouse, an NOG (NOD/gc^{-/-}) mouse, a Rag-1 (rag-1^{-/-}/gc^{-/-}) mouse, or a Rag-2(rag-2^{-/-}/gc^{-/-}) mouse. In some embodiments, the non-human animal host (e.g. mouse, guinea pigs, rabbits, and pigs) is sub-lethally irradiated prior to introduction of the heterologous hematopoietic stem cells. In some embodiments, the mouse is irradiated with about 300 Rads.

In some embodiments, the invention provides methods for producing a xenochimaeric non-human animal (e.g. mouse, guinea pigs, rabbits, and pigs) comprising providing HSC to the non-human animal, wherein the HSCs are from a heterologous animal (e.g. a domesticated animal, a wild animal, a human companion animal, a zoo animal, a farm animal). In some embodiments, the non-human animal is a canine (e.g. dog) or a feline (e.g. cat). In some embodiments, the HSCs are derived from a blood sample, a bone marrow sample, or a cord blood sample from the heterologous animal or from the heterologous species. In some embodiments, the HSCs are CD34+ cells. In some embodiments, the HSCs are CD34⁺/CD38^{lo}/CD150⁺/CD48^{lo}/lin⁻ cells. In some embodiments, the heterologous HSCs are conditionally immortalized HSCs. In some embodiments, the heterologous HSCs have been conditionally immortalized by culturing the HSCs in the presence of a MYC polypeptide and a BCL-2 polypeptide. In some embodiments, the MYC is a MYC-ER. In some embodiments, the MYC polypeptide and/or the BCL-2 polypeptide are fused to a peptide that enhances cellular uptake of the polypeptide. In further embodiments, the peptide that enhances the uptake of the polypeptide is a Tat peptide. In some embodiments, the Tat peptide is RKKRRQRRR. In yet further embodiments, the MYC polypeptide is a Tat-MYC polypeptide and/or the BCL-2 polypeptide is a Tat-BCL-2 polypeptide. In other embodiments, the heterologous HSCs have been conditionally immortalized by introducing nucleic acids encoding myc and/or bcl-2 to the cell. In some embodiments, the nucleic acid encoding myc is a nucleic acid encoding MYC-ER. In some embodiments, the nucleic acids were introduced to the cell using one or more integrating viral vectors.

In some embodiments, the invention provides methods for producing a xenochimaeric non-human animal (e.g. mouse, guinea pigs, rabbits, and pigs) comprising providing HSC from a heterologous animal and a tumor from the heterologous animal to the non-human animal. In some embodiments, the tumor sample is introduced to the non-human animal (e.g. mouse, guinea pigs, rabbits, and pigs) by engrafting the tumor subcutaneously, orthotopically or by a hematogenous route. In some embodiments, the solid tumor sample is one or more of a head and neck tumor, a brain tumor, an eye tumor, a thyroid tumor, an adrenal tumor, a salivary gland tumor, an esophageal tumor, a gastric tumor, an intestinal tumor, a colon tumor, a lung tumor, a breast tumor, a liver tumor, a pancreas tumor, a kidney tumor, a bladder tumor, a prostate tumor, a muscular tumor, an osseous tumor, a skin tumor, or a stroma/sarcoma tumor.

In some aspects, the invention provides methods for producing a population of xenochimaeric non-human animals (e.g. mice, guinea pigs, rabbits, and pigs), the method comprising a) introducing heterologous HSCs to each individual in the population, and b) introducing a portion of a malignant or benign tumor from the heterologous animal (or from the heterologous species) to each individual in the population. In some embodiments, the heterologous animal is a non-human animal (e.g. a domesticated animal, a wild animal, a human companion animal, a zoo animal, a farm animal). In some embodiments, the non-human animal is a canine (e.g. dog) or a feline (e.g. cat). In some embodiments, the heterologous animal is a human. In some embodiments, the HSCs and the tumor are from the same individual.

In some embodiments, the invention provides methods for producing a population of xenochimaeric non-human animals (e.g. mice, guinea pigs, rabbits, and pigs) wherein the non-human animals are immunodeficient mice. In some embodiments, the immunodeficient non-human animals lack one or more of T cells, NKT cells, B cells and NK cells. In some embodiments, the immunodeficient mice are a nu⁻/nu⁻ mice, NSG (NOD/SCID/gc^{-/-}) mice, NOG (NOD/gc^{-/-}) mice, Rag-1 (rag-1^{-/-}/gc^{-/-}) mice, or Rag-2(rag-2^{-/-}/gc^{-/-}) mice. In some embodiments, the non-human animals are sub-lethally irradiated prior to introduction of the heterologous hematopoietic stem cells. In some embodiments, the mice are irradiated with about 300 Rads.

In some embodiments, the invention provides methods for producing a population of xenochimaeric non-human animals (e.g. mice, guinea pigs, rabbits, and pigs) comprising providing HSC from a heterologous animal to the non-human animal host, wherein the HSCs are derived from a blood sample, a cord blood sample, or a bone marrow sample from the heterologous animal or the heterologous species (e.g. a domesticated animal, a wild animal, a human companion animal, a zoo animal, a farm animal). In some embodiments, the heterologous non-human animal is a canine (e.g. dog) or a feline (e.g. cat). In some embodiments, the HSCs are CD34+ cells. In some embodiments, the HSCs are CD34⁺/CD38^{lo}/CD150⁺/CD48^{lo}/lin⁻ cells. In some embodiments, the heterologous HSCs are conditionally immortalized HSCs. In some embodiments, the heterologous HSCs have been conditionally immortalized by culturing the HSCs in the presence of a MYC polypeptide and a BCL-2 polypeptide. In some embodiments, the MYC polypeptide and/or the BCL-2 polypeptide are fused to a peptide that enhances cellular uptake of the polypeptide. In further embodiments, the peptide that enhances the uptake of the polypeptide is a Tat peptide. In yet further embodiments, the MYC polypeptide is a Tat-MYC polypeptide and/or the BCL-2 polypeptide is a Tat-BCL-2 polypeptide. In other embodiments, the heterologous HSCs have been conditionally immortalized by introducing nucleic acids encoding myc and/or bcl-2 to the cell. In some embodiments, the nucleic acids were introduced to the cell using one or more integrating viral vectors.

In some embodiments, the invention provides methods for producing a population of xenochimaeric non-human animals (e.g. mice, guinea pigs, rabbits, and pigs) comprising providing HSC from a heterologous animal and a portion of a tumor from the heterologous animal or heterologous species (e.g. a domesticated animal, a wild animal, a human companion animal, a zoo animal, a farm animal). In some embodiments, the heterologous non-human animal is a canine (e.g. dog) or a feline (e.g. cat). In some embodiments, the tumor sample is introduced to the non-human animal host (e.g. mice, guinea pigs, rabbits, and pigs) by engrafting the tumor subcutaneously, orthotopically or by a hematogenous route. In some embodiments, the tumor sample is a solid tumor sample. In some embodiments, the solid tumor is from one or more of a head and neck tumor, a brain tumor, an eye tumor, a thyroid tumor, an adrenal tumor, a salivary gland tumor, an esophageal tumor, a gastric tumor, an intestinal tumor, a colon tumor, a lung tumor, a breast tumor, a liver tumor, a pancreas tumor, a kidney tumor, a bladder tumor, a prostate tumor, a muscular tumor, an osseous tumor, a skin tumor, or a stroma/sarcoma tumor.

In some aspects, the invention provides methods for evaluating a test agent for treating cancer, the method comprising a) administering the test agent to xenochimaeric non-human animals (e.g. mice, guinea pigs, rabbits, and pigs) in a population of xenochimaeric non-human animals (e.g. mice, guinea pigs, rabbits, and pigs), comprising heterologous HSCs and a portion of a heterologous tumor, as described in any of the embodiments described above or herein, and b) evaluating the response of the tumor to the test agent. In some embodiments, the test agent is administered to the xenochimaeric mice about two to about four weeks following introduction fo the tumor to the mice. In some embodiments, the test agent is administered to the xenochimaeric mice after one or more of formation of heterologous stroma, or reversions towards one or more tumor phenotype or genotype. In some embodiments, the evaluation of the response of the tumor to the test agent is an evaluation of the size of the tumor wherein a decrease in the size of the tumor indicates therapeutic efficacy. In some embodiments the evaluation of the response of the tumor to the test agent is an evaluation of the growth rate of the tumor wherein a decrease in the growth rate of the tumor indicates therapeutic efficacy. In some embodiments, the evaluation of the response of the tumor to the test agent is an evaluation of the vascularization of the tumor wherein a decrease in the vascularization of the tumor indicates therapeutic efficacy. In some embodiments, the evaluation of the response of the tumor to the test agent is an evaluation of stromal cells from the heterologous animal in the tumor wherein a decrease in the number of stromal cells or a decrease in a lineage of stromal cells from the heterologous animal in the tumor indicates therapeutic efficacy. In some embodiments, the evaluation of the response of the tumor to the test agent is an evaluation of stromal cells from the heterologous animal in the tumor wherein an increase in the number of stromal cells or an increase in a lineage of stromal cells from the heterologous animal in the tumor indicates therapeutic efficacy. In some embodiments, the evaluation of the response of the tumor to the test agent is an evaluation of stromal cells from the heterologous animal in the tumor wherein a change in the number of stromal cells, a change in a lineage of stromal cells, or a change in the relative ratios of lineages of stromal cells from the heterologous animal in the tumor indicates therapeutic efficacy. In some embodiments, the evaluation of the response of the tumor to the test agent is an evaluation of immune cells from the heterologous animal in the tumor wherein an increase in the number of immune cells or an increase in a lineage of immune cells from the heterologous animal in the tumor indicates therapeutic efficacy. In some embodiments, the evaluation of the response of the tumor to the test agent is an evaluation of immune cells from the heterologous animal in the tumor wherein a decrease in the number of immune cells or a decrease in a lineage of immune cells from the heterologous animal in the tumor indicates therapeutic efficacy. In some embodiments, the evaluation of the response of the tumor to the test agent is an evaluation of immune cells from the heterologous animal in the tumor wherein a change in the number of immune cells, a change in a lineage of immune cells, or a change in the relative ratios of lineages of immune cells from the heterologous animal in the tumor indicates therapeutic efficacy. In some embodiments, the evaluation of the response of the tumor to the test agent is an evaluation of the survival of the xenochimaeric mice bearing tumors, wherein an increase in the survival of the xenochimaeric mice bearing tumors indicates therapeutic efficacy. In some embodiments, the evaluation of the response of the tumor to the test agent is an evaluation of one or more of the embodiments described above.

In some aspects, the invention provides methods for evaluating the stroma of a tumor sample, the method comprising a) removing the tumor from a xenochimaeric non-human animal (e.g. mouse, guinea pigs, rabbits, and pigs), comprising heterologous HSCs and a portion of a heterologous tumor, as described in any of the embodiments above or herein, and b) detecting the presence of stroma from the heterologous animal in the tumor. In some embodiments, the stroma is one or more of T cells, B cell, macrophages, dendritic cells, NK cells, NKT cells, neutrophils, basophils, endothelial cells, epithelial cells. In some embodiments, the detecting the presence of stroma from the heterologous animal is by histochemistry. In some embodiments, the detecting the presence of stroma from the heterologous animal is by FISH. In some embodiments, the detecting the presence of stroma from the heterologous animal is by fluorescence activated cell sorting (FACS). In some embodiments, the detecting the presence of stroma from the heterologous animal is by detecting nucleic acid specific for the stroma from the heterologous animal. In some embodiments, the detecting the presence of stroma from the heterologous animal is a detection of the activity of the stroma from the heterologous animal. In some embodiments, the activity is secretion of stromal factors; for example but not limited to SDFl and HGF. In some embodiments, the activity of the stroma is measured in response to administration of a test agent to the xenochimaeric non-human animal (e.g. mouse, guinea pigs, rabbits, and pigs). In some embodiments, the agent is an anti-cancer agent or a candidate anti-cancer agent. In some embodiments, the xenochimaeric non-human animal (e.g. mouse, guinea pigs, rabbits, and pigs) comprises human HSCs. In some embodiments, the presence of heterologous (e.g. non-mouse) engrafted cells CD151⁺, CD31⁺,1y1⁺, CD 45⁺, CD3⁺, CD19⁺, CD68⁺, CD4⁺, SMA⁺, and/or CD57⁺ from the heterologous animal indicates the presence of stroma from the heterologous animal.

In some aspects, the invention provides methods of treating a cancer patient, the method comprising administering an effective amount of an anticancer agent to the cancer patient, wherein the anticancer agent was shown to be effective in delaying or inhibiting the growth of a tumor in one or more xenochimaeric non-human animal (e.g. mice, guinea pigs, rabbits, and pigs) comprising HSCs and a tumor sample from the patient as described in any of the above embodiments or herein. In some embodiments, the invention provides methods of treating a cancer patient, the method comprising administering an effective amount of an anticancer agent to the cancer patient, wherein the anticancer agent was shown to be effective in delaying or inhibiting the growth a tumor in a population of xenochimaeric non-human animals (e.g. mice, guinea pigs, rabbits, and pigs) prepared with HSCs and a tumor sample from the patient as described in any one of the above embodiments or herein.

In some aspects, the invention provides methods of treating cancer in a patient, the method comprising administering an effective amount of an effective anticancer agent to the cancer patient, wherein the anticancer agent had been shown to be effective in delaying or inhibiting the growth of the tumor in a xenochimaeric non-human animal (e.g. mouse, guinea pigs, rabbits, and pigs) according to the method comprising a) introducing HSCs from the patient to the mouse, b) introducing a portion of malignant tumor from the patient to the mouse, c) administering a candidate anticancer agent to the mouse, d) analyzing the xenochimaeric mice for effective anticancer activity, wherein an effective anticancer agent is one that delays or inhibits the growth of the tumor in the xenochimaeric mouse compared to the growth of a tumor in a xenochimaeric mouse that was not treated with a candidate anticancer agent. In some embodiments, the mouse is an immunodeficient mouse. In further embodiments, the immunodeficient mouse lacks one or more of T cells, NKT cells, B cells and NK cells. In yet further embodiments, the immunodeficient mouse is a nu⁻/n⁻ mouse, an NSG (NOD/SCID/gc^{-/-}) mouse, an NOG (NOD/gc^{-/-}) mouse, a Rag-1 (rag-1^{-/-}/gc^{-/-}) mouse, or a Rag-2 (rag-2^{-/-}/gc^{-/-}) mouse. In some embodiments, the mice are sub-lethally irradiated prior to introduction of the heterologous hematopoietic stem cells. In some embodiments, the mice are irradiated with about 300 Rads.

In some aspects, the invention provides methods of treating cancer in a patient, the method comprising administering an effective amount of an effective anticancer agent to the cancer patient, wherein the anticancer agent had been shown to be effective in delaying or inhibiting the growth of the tumor in a xenochimaeric mouse comprising HSC from a heterologous animal. In some embodiments, the HSCs are derived from a blood sample, a cord blood sample, or a bone marrow sample from the heterologous animal or the heterologous species. In some embodiments, the HSCs are CD34⁺ cells. In some embodiments, the HSCs are CD34⁺/CD38^{lo}/CD150⁺/CD48^{lo}/lin⁻ cells. In some embodiments, the heterologous HSCs are conditionally immortalized HSCs. In some embodiments, the heterologous HSCs have been conditionally immortalized by culturing the HSCs in the presence of a MYC polypeptide and a BCL-2 polypeptide. In some embodiments, the MYC polypeptide and/or the BCL-2 polypeptide are fused to a peptide that enhances cellular uptake of the polypeptide. In further embodiments, the peptide that enhances the uptake of the polypeptide is a Tat peptide. In yet further embodiments, the MYC polypeptide is a Tat-MYC polypeptide and/or the BCL-2 polypeptide is a Tat-BCL-2 polypeptide. In other embodiments, the heterologous HSCs have been conditionally immortalized by introducing nucleic acids encoding myc and/or bcl-2 to the cell. In some embodiments, the nucleic acids were introduced to the cell using one or more integrating viral vectors.

In some aspects, the invention provides methods of treating cancer in a patient, the method comprising administering an effective amount of an effective anticancer agent to the cancer patient, wherein the anticancer agent had been shown to be effective in delaying or inhibiting the growth of the tumor in a xenochimaeric mouse comprising HSC from a heterologous animal and a tumor from the heterologous animal. In some embodiments, the tumor sample is introduced to the mice by engrafting the tumor subcutaneously, orthotopically or by a hematogenous route. In some embodiments, the tumor sample is a solid tumor sample. In some embodiments, the solid tumor is from one or more of a head and neck tumor, a brain tumor, an eye tumor, a thyroid tumor, an adrenal tumor, a salivary gland tumor, an esophageal tumor, a gastric tumor, an intestinal tumor, a colon tumor, a lung tumor, a breast tumor, a liver tumor, a pancreas tumor, a kidney tumor, a bladder tumor, a prostate tumor, a muscular tumor, an osseous tumor, a skin tumor, or a stroma/sarcoma. In some embodiments, the cancer is a head and neck cancer, a melanoma, a brain cancer, a respiratory tract cancer, an endocrine cancer, a breast cancer, a prostate cancer, a colorectal cancer, a gastrointestinal cancer, an osteosarcoma, a myeloblastoma, acute lymphoblastic leukemia (ALL), acute myelogenous leukemia (AML), chronic lymphocytic leukemia (CLL), chronic myelogenous leukemia (CML), or non-Hodgkin's lymphoma (NHL).

In some embodiments, the invention provides methods of treating cancer in a patient, the method comprising administering an effective amount of an effective anticancer agent to the cancer patient, wherein the anticancer agent had been shown to be effective in delaying or inhibiting the growth of the tumor in a xenochimaeric mouse comprising HSC from a heterologous animal and a tumor from the heterologous animal wherein the HSCs are introduced to the mice about 8 to about 10 weeks prior to introduction of the tumor to the mice. In some embodiments, the candidate anticancer agent is administered to the xenochimaeric mice about two to about four weeks following introduction of the tumor to the mice. In some embodiments, the anticancer agent is a small molecule, a polypeptide, a nucleic acid, an antibody, a monoclonal antibodies conjugated to one or more toxins, a decoy receptor, a gene-mediated therapy, a natural immune modulator, a synthetic immune modulator, a vaccine or a radiotherapy. In some embodiments, the anticancer agent is used in combination with a chemotherapy, a radiotherapy and/or an immune therapy.

In some embodiments, the heterologous HSCs provided to the non-human animals (e.g. mice) include genetic modifications, for example definitive, transient or inducible. In some embodiments, the genetic modifications modify, suppress or enhance the expression of biologic molecules including, but not limited to, DNA, RNA, miRNA or protein following engraftment into the non-human animal. In some embodiments, the genetic modifications inhibit the expression or activation of genes that are relevant in the stroma to tumor interaction. In some embodiments, the genetic modifications include the insertion of vectors carrying short hairpin RNA (shRNA) inhibiting one or more of WNT7A, WNT4, WNT10A, WNT3A, WNT7B, WNT6, WNT16, WNT11, WNT9A, WNT5B, CSNK1E, AXIN1, DVL1, TCF3, MYC, JUN, MMP9, MMP10, MMP11, MMP12, MMP15, MMP17, MMP19, CLDN7, CLDN4, CLDN14, CLDN1, CLDN22, CLDN15, SNAIL TWIST1, or VIM. These genetic modifications may be utilized in models including but not limited to drug development, assay development, and biology development.

In some aspects, the invention provides a xenochimaeric mouse comprising conditionally immortalized HSCs from a heterologous animal and a solid tumor from the heterologous animal.

### BRIEF DESCRIPTION OF THE DRAWINGS

FIG. 1 shows examples of tumors from standard (nu/nu) implantation models (F2) and original patient tumors (F0) as reference.

FIG. 2 shows a silver stained 15% SDS-PAGE gel with purified recombinant Tat-MYC (lane 1) and Tat-Bcl-2 (lane 2) fusion proteins. Molecular weight markers are shown in lane 3.

FIG. 3 shows graphs demonstrating the purified recombinant Tat-MYC and Tat-Bcl-2 fusion proteins are biologically active. The scatter plots show example FACS analysis of CD4⁺ T-cells activated by antibodies to CD3 and CD28, and subsequent incubation with Tat-MYC and Tat-Bcl-2 in fresh media. This is compared to scatter plots of a control with either no Tat-MYC or no Tat-Bcl-2 present. The graphs below show the percentage of living T-cells with either a constant amount (50 μg/mL) of Tat-MYC and an increasing amount of Tat-Bcl-2 (left panel), or a constant amount (50 μg/mL) of Tat-Bcl-2 and an increasing amount of Tat-MYC (right panel).

FIG. 4 shows a FACS analysis demonstrating the expansion of lt-HSCs (indicated by CD34⁺, CD38^{lo} profile) after incubation with recombinant purified Tat-MYC and Tat-Bcl-2 for 28 days.

FIG. 5 is a graph showing the magnitude of expansion in vitro of human lt-HSCs using recombinant purified Tat-MYC and Tat-Bcl-2. The total number of cultured human lt-HSCs was counted by FACS as demonstrated by the CD34⁺ marker during the course of expansion.

FIG. 6 shows scatter plots of FACS analysis used to assess mouse blood for human CD45⁺ and CD3⁺ cells with (right panel) and without (left panel) implantation of ctlt-HSCs into a NSG mouse.

FIG. 7a shows images of nu/nu, NSG and xenochimaeric mice implanted with early passage (F2) human tumors. FIG. 7b shows the growth of implanted human tumors (CUHN004 and CUHN013) after two months for each mouse strain. Data shown are averages of two subsequent experiments.

FIG. 8 shows FACS analysis of cancer stem cells (CSCs) with CD44, CD24 and aldehyde dehydrogenase 1 (ALDH) markers comparing nu/nu, NSG and xenochimaeric mice implanted with early passage (F2) human tumors.

FIG. 9 shows the FACS analysis demonstrating the percentage of human CD151⁺ cells within the implanted tumor from nu/nu, NSG and xenochimaeric mice one and three months after implantation of the early passage (F2) human tumors.

FIG. 10 shows a cot assay comparison between nu/nu, NSG and xenochimaeric mouse implanted with CUHN013 tumors. Images are from a differential fluorescence in situ hybridization (FISH) DNA staining assay of human tumors implanted in nu/nu, NSG and xenochimaeric mice.

FIG. 11 shows the results of a DNA fingerprinting assay using human thyroid peroxidase (TPDX, left panel) or von Willebrand Factor type A (vWA) (right panel) loci small tandem repeat (STR) elements from previously isolated mouse genomic DNA (lane 1), genomic DNA from the CUHN013 tumor prior to implantation in a xenochimaeric mouse (lane 2) and DNA isolated from human CD151⁺ cells from peripheral blood of a xenochimaeric mouse implanted with the same early passage F2 human tumor but immune system from a differing human donor.

FIG. 12 shows FACS analysis of peripheral mouse blood assed for the presence of human CD45⁺, CD3⁺ cells with (xenochimaeric mice, right panel) and without (NSG control, left panel) implantation of ctlt-HSCs originating from a chemotherapy patient into a NSG mouse.

FIG. 13 shows immunofluorescence images of the CUHN004 tumor taken from the patient (left panel), implanted on NGS (middle panel) or xenochimaeric mouse (right panel) stained with CD151 antibodies, demonstrating recuperation of stromal elements.

FIG. 14 shows the in vitro characterization of stromal cells. Analysis by flow cytometry indicates that cells containing the human CD45⁺/CD151⁺ antigens (boxed) are not present in tumors removed from nude and NSG mice but compose over 10% of the gated cells examined from this Xenochimaeric mice.

FIG. 15 shows flow cytometry of additional tissues demonstrating that human CD45⁺/151⁺ cells are present in the bone marrow, spleen, and blood of the tumor-bearing Xenochimaeric mice, but are absent in nude and NSG mice.

FIG. 16 shows the presence of human stroma in Xenochimaeric mice. Human cells invade the tumors in the Xenochimaeric mice, but no evidence of invasion can be seen in the nude or NSG mice. FIG. 16a provides a bioanalyzer gel of the PCR analysis of two well-defined STR loci, using primers originally constructed for forensic examination. Patient DNA is from the tumor from which xenografts were obtained (F0, lanes 2 and 6). Xenochimaeric mice intratumor CD45⁺/CD151⁺ cell DNA was obtained by cell sorting of a CUHN004 tumor grown on Xenochimaeric mice (X, lanes 3 and 7). FIGS. 16 b and e shows patterns of human CD151 immunofluorescence. In NSG tumors (b), the stroma remains unstained, while in Xenochimaeric mice (c) the unstained mouse stroma is punctuated with CD151⁺ human cells. Magnification is 20× and the scale bar equals 50 μm. FIGS. 16d and e show FISH analysis of nude and Xenochimaeric mice tumors, using species-specific Cot-1 probes. Slides of these tumors were H/E stained, and the corresponding region is shown below. Magnification is 10× for the tumor sections and 20× for the enlarged portions, and the scale bars equal 50 μm. FIGS. 16f and g show FISH analysis images of tumor sections using fluorescently-labeled X (red) and Y (green) probes (NSG (f) and Xact Mice (g). Slides of these tumors were H/E stained and the corresponding region is shown. A dashed line has been added to demarcate the approximate tumor-stroma boundary in these images. Detail inserts were captured under increased magnification (100×). In all images, the scale bar equals 50 μm.

FIG. 17 shows the characterization human stromal cells within Xenochimaeric mouse tumors by comparing CUHN004 and CUHN013 patient (F0) tumors with their corresponding NSG and Xenochimaeric mice xenografts. FIG. 17a shows H/E comparisons of the F0, NSG, and Xenochimaeric mice specimens from both tumors. FIG. 17b shows IHC using the human CD45 antibody (red) in all specimens for both tumors. FIGS. 17c-h show that in NSGs, no human cells are present, while in Xenochimaeric mice distinct populations of cells with either or both (indicated with red arrows) surface markers are present in patterns reminiscent of those seen in F0 tumors. FIG. 17c shows tumor IHC using both human (red) and mouse (brown) CD45 antibodies. FIG. 17d shows staining using dual human CD3 (brown) and CD45 (red) IHC. FIG. 17e shows staining using dual human CD19 (brown) and CD45 (red) IHC. FIG. 17f shows staining using dual human CD68 (brown) and CD45 (red) IHC. FIG. 17g shows staining using dual human aSMA (brown) and CD45 (red) IHC. FIG. 17h shows staining for human CD4 IHC. Magnification is 40× and the scale bar equals 50 μm.

FIG. 18 shows RNA sequences generated from high-throughput sequencing aligned to the human genome (NCBI 37.2). Resultant unaligned sequences were then aligned to the mouse genome (NCBI 37.2), and any remaining sequences were classified as unmatched, most likely due to base repetitivity or sequencing errors.

FIG. 19 shows results of deep RNA sequencing. FIG. 19 shows waterfall graphs showing the relative enrichment of all GO terms associated with the differentially expressed genes identified in the F0 and Xenochimaeric mice CUHN004 and CUHN013 tumors. The frequency with which GO terms were associated with differentially expressed genes was used to calculate a GSEA score, based on the probability that multiple identified genes would be associated with a single process. Enrichment scores greater than 1.3 indicate that the GO term is statistically enriched (P-value<0.05) among these genes. After this enrichment analysis, GO terms were coded according to their overarching biological process, the most frequently observed of which were immune system, extracellular matrix (ECM), and epithelial mesenchymal transition (EMT). A paired z-test for proportions (inset table) shows that the enrichment of the GO terms representing each of these processes is statistically significant in genes differentially expressed in the F0 and Xenochimaeric mice tumors. FIG. 19(d) shows an enlargement of the top twenty most enriched GO terms in the CUHN004 and CUHN013 tumor waterfall graphs from above.

FIG. 20 provides analysis of overexpressed Xenochimaeric mice genes. Genes identified by RNA sequencing from the CUHN004 or CUHN013 tumors which are expressed at least five-fold (32 times) more abundantly in the Xenochimaeric mice than in the F0, NSG, or nude mouse samples.

FIG. 21 identifies enriched processes among overexpressed CUHN004 Xenochimaeric mice genes. The genes overexpressed in CUHN004 Xenochimaeric mice are statistically enriched with cytokine pathway components. Their enrichment score is calculated by the NIH-DAVID algorithm and derived from the negative log of the P-value of their presence together within the queried gene list. Any enrichment score greater than 1.3 correlates with a P-value of less than 0.05. No enrichment was identified for the genes overexpressed in CUHN013 Xenochimaeric mice.

FIG. 22 provides differentially expressed genes. These genes in the CUHN004 and CUHN013 tumors were either calculated by Cuffdiff to be differentially expressed in the Xenochimaeric mice and F0 samples, or they were subjected to an expression fold-change analysis between the Xenochimaeric mice-F0 and the NSG-nude groups and found to have an absolute fold change value ≧2.

FIG. 23 provides enriched processes among differentially expressed genes. The differentially expressed genes in the Xenochimaeric mice and F0 samples for each tumor are statistically enriched with members of several different biological processes. Their enrichment score is calculated by the NIH-DAVID algorithm and derived from the negative log of the P-value of their presence together within the queried gene list. Any enrichment score greater than 1.3 correlates with a P-value of less than 0.05.

FIG. 24 provides activated genes in Xenochimaeric mice tumors. These genes were identified from RNA sequencing data from their low expression in the nude and NSG tumors and dramatically increased expression in F0 and Xenochimaeric mice tumors. To be considered activated, a gene's expression in the Xenochimaeric mice tumor must be greater than four times its expression in the nude or NSG tumors. Additionally, its average expression in F0 and Xenochimaeric mice tumors must be greater than 20 times its average expression in the nude and NSG tumors. The activated genes highlighted in pink are implicated in ECM function. Those in green have a known role in EMT, while those in blue play a role in the immune response. Genes activated in both tumors are in bold.

FIG. 25 provides gene enrichment groups. The activated genes identified in the xenochimaeric mice tumors are statistically enriched with members of immune response, inflammation, and cell adhesion pathways. The enrichment score is calculated by the NIH-DAVID algorithm and derived from the negative log of the P-value of their presence together within the queried gene list. Any enrichment score greater than 1.3 correlates with a P-value of less than 0.05.

FIG. 26 demonstrates tumor growth during and after radiation therapy. After receiving a fractionated 12 Gy dose of radiation, only tumors in the Xenochimaeric mice regressed. Tumors in the nude and NSG mice grew at the same rate as their non-irradiated control tumors.

FIG. 27 shows irradiated Xenochimaeric mice flank and OOF tumors express increased CXCL16. IHC using a CXCL16 antibody indicates that CUHN004 tumors in both NSG and Xenochimaeric mice express low levels of CXCL16. However, after 12 Gy of irradiation, only the Xenochimaeric mice flank and OOF tumors increase their expression of this chemokine.

FIG. 28 shows RT prompts Xenochimaeric mice flank and OOF tumor invasion by cytotoxic T-cells and NK cells. Dual CD3 and CD45 IHC identifies T-cells in Xenochimaeric mice tumors (left panels). Dual CD8 and CD45 IHC identifies cytotoxic T-cells, present in much higher abundance in flank tumors of irradiated Xenochimaeric mice (right panels).

FIGS. 29A, 29B, 29C, 29D, and 29E show an illustrative embodiment of a graphical representation of the expansion of human cord blood cell-derived HSCs with Tat-Myc and Tat-Bcl-2. FIG. 29A shows an illustrative embodiment of a graphical representation of a FACS analysis of the surface phenotype of the human cord blood cells expanded in vitro for 14 days (Top panels cytokine cocktail only; Bottom panels cytokine cocktail supplemented with Tat-Myc and Tat-Bcl-2). FIG. 29B shows an illustrative embodiment of a graphical representation of the kinetics of CD34⁺ cells expansion in vitro under both sets of conditions.

FIG. 29C shows an illustrative embodiment of the images of three different colony types developed in methylcellulose assays under conditions that support myeloerythroid differentiation, derived from human ptlt-HSCs. FIG. 29D shows an illustrative embodiment of a graphical representation of the quantification of each colony type that was observed in methylcellulose cultures seeded with either 10³ cord blood cells cultured with a cytokine cocktail (FCB), 10³ cord blood cells cultured with a cytokine cocktail supplemented with Tat-Myc and Tat-Bcl-2 (FCB+TMTB), or 10⁴ fresh un-manipulated cord blood cells (10⁴ Fresh FCB). FIG. 29E shows an illustrative embodiment of a graphical representation of the quantification of the number of colonies observed in methylcellulose cultures upon replating of the cells shown in FIG. 29D.

FIGS. 30A, 30B, 30C, 30D, 30E, 30F, and 30G show an illustrative embodiment of a graphical representation of the functional analysis of human cord blood derived protein-transduced long term (ptlt)-HSC in vivo. FIG. 30A shows an illustrative embodiment of a graphical representation of a FACS analysis of the bone marrow of cohorts of sublethally irradiated NSG mice given transplants of 10⁶ cord blood cells expanded in vitro in a cocktail of cytokines (first panel; FCB), or expanded in a cocktail of cytokines supplemented with Tat-Myc and Tat-Bcl-2 (second panel; FCB TMTB), or 5×10⁶ fresh un-manipulated cord blood cells (third panel; Fresh FCB). FIG. 30B shows an illustrative embodiment of a graphical representation of a FACS analysis of bone marrow, spleen and thymus cells from the xenochimaeric mice. All cells were stained for human CD45. Gating on CD45⁺ cells showed human CD34⁺ CD38¹° cells in the bone marrow (first panel; BM); human CD19⁺ and human CD3⁺ lymphocytes in the spleen (second panel; spleen); and human CD3⁺ cells in the thymus (third panel; thymus). FIG. 30C shows an illustrative embodiment of a graphical representation of a FACS analysis of human splenic B-cells labeled with CFSE and cultured in the presence of monoclonal antibodies to human CD40 and IgM. Human B-cells that developed in NSG xenochimaeric mice underwent proliferation following stimulation of their antigen receptor.

FIG. 30D shows an illustrative embodiment of a graphical representation of the quantification of myeloerythroid colonies from human CD34⁺ CD38^{lo} cells obtained from the bone marrow of NSG xenochimaeric mice and plated on methycellulose. FIG. 30E shows an illustrative embodiment of a graphical representation of the quantification of the development of myeloerythroid colonies following replating. FIG. 30F shows an illustrative embodiment of a graphical representation of the quantification of myeloid and lymphoid cell differentiation (CD llb, CD33, CD3, and CD19 expression) in the CD45 positive population of bone marrow cells expanded in vitro in a cocktail of cytokines (open circles) or a cocktail of cytokines supplemented with Tat-Myc and Tat-Bcl-2 (black squares). FIG. 30G shows an illustrative embodiment of a graphical representation of the quantification of myeloid and lymphoid cell differentiation (CD llb, CD33, CD3, and CD19 expression) in the CD45 positive population of spleen cells expanded in vitro in a cocktail of cytokines (open circles) or a cocktail of cytokines supplemented with Tat-Myc and Tat-Bcl-2 (black squares).

FIGS. 31A, 31B, 31C, 31D, 31E, 31F and 31G show an illustrative embodiment of a graphical representation of the expansion of adult human G-CSF mobilized HSCs in vitro with Tat-Myc and Tat-Bcl-2. FIG. 31A shows an illustrative embodiment of a graphical representation of the surface phenotype of human CD45⁺ cells showing an enrichment of the human CD34⁺ and CD38⁺ fraction. FIG. 31B shows an illustrative embodiment of a graphical representation of the kinetics of cell expansion in vitro over 18 days in culture in the presence of Tat-Myc and Tat-Bcl-2. FIG. 31C shows an illustrative embodiment of a graphical representation showing that 5×10³ human adult G-CSF HSCs, expanded in vitro with Tat-Myc and Tat-Bcl-2, gave rise to 4 morphologically distinct colony types in methylcellulose. FIG. 31D shows an illustrative embodiment of a graphical representation of FACS analysis showing that human adult G-CSF HSCs expanded in vitro with Tat-Myc and Tat-Bcl-2 gave rise to human hematopoietic lineages in xenochimaeric NSG mice. Bone marrow was from NSG mice transplanted ptlt-HSCs expanded with a cytokine cocktail supplemented with Tat-Myc and Tat-Bcl-2 (first panel; G-CSF+TMTB) or with fresh un-manipulated cord blood cells (second panel; Fresh FCB). FIG. 31E shows an illustrative embodiment of a graphical representation of FACS analysis of cells from bone marrow, spleen, and thymus. Bone marrow cells included human CD45 cells that were also human CD34⁺ and CD38⁺ (first panel), spleen cells included human CD45 cells that also stained for human CD3 (second panel), and thymus cells included human CD45 cells as well as CD3 (third panel). FIGS. 31F and 31G show an illustrative embodiment of a graphical representation of a cohort of xenochimaeric mice engrafted with 10⁶ G-CSF mobilized cells expanded in vitro in a cocktail of cytokines supplemented with Tat-Myc and Tat-Bcl-2 (black squares) were assessed for myeloid and lymphoid cell differentiation. The CD45 positive population of bone marrow cells (FIG. 31F) and spleen cells (FIG. 31G) were analyzed for CD 11b, CD33, CD3, and CD19 expression.

FIG. 32 shows an illustrative embodiment of a graphical representation of a FACS analysis of mouse splenic T-cells and B-cells labeled with CFSE and cultured in the presence of monoclonal antibodies to mouse CD3 or CD40 and IgM, respectively. Mouse T-cells (light-gray left-most line, first panel) and B-cells (light-gray left-most line, second panel) that developed in Rage^{-/-} mice transplanted with expanded HSC from 5FU treated C57BL.6 underwent proliferation following stimulation of their antigen receptor compared to unstimulated cells (dark gray right-most line).

### DETAILED DESCRIPTION OF THE INVENTION

The present invention provides methods for producing xenochimaeric mice by engrafting paired tumor and immortalized bone marrow precursors from the same patient or the same species in an immunodeficient non-human animal host (e.g. mouse model). In some embodiments, the tumor is malignant. In some embodiments, the tumor is benign. As such, the model replicates the originator tumor. In some aspects of the invention, xenochimaeric mice are used to model tumor biology, to recapitulate disease pathogenesis in vivo, to test and/or model therapeutic treatments, to assess efficacy and modes of action of test agents, etc. In some embodiments, the model focuses on for example, interactions between stromal cells and tumor cells within the tumor. In other aspects, xenochimaeric mice of the invention are used for drug discovery. For example, xenochimaeric mice of the invention can be used to evaluate drugs and other treatments that specifically target the stroma of a tumor. In some aspects of the invention, xenochimaeric mice are used in a method of treatment where one or more xenochimaeric mice are generated using hematopoietic stem cells (HSCs) from a patient and a sample of a tumor from the same patient (or the same species). Candidate drugs are then screened for therapeutic efficacy using the patient-specific xenochimaeric mouse.

### I. DEFINITIONS

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

The terms “polypeptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. The terms polypeptide and protein also encompass fragments of full-length polypeptide or protein, unless clearly indicated otherwise by context.

The terms “nucleic acid molecule” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The term “isolated” as used herein refers to a molecule that has been separated from at least some of the components with which it is typically found in nature. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated” so long as that polynucleotide is not found in that vector in nature.

“MYC,” “c-MYC,” or “v-myc myelocytomatosis viral oncogene homolog” as used herein refers to a native MYC from any vertebrate source, including mammals such as primates (e.g., humans) and rodents (e.g., mice and rats), unless otherwise indicated. The terms encompass the genomic location, (e.g., 8q24 cytogenetic band, chromosome 8:128747680-128753680, and/or GC08P128748), “full-length,” unprocessed MYC as well as any form of MYC that result from processing in the cell. The terms also encompass naturally occurring variants of MYC, e.g., splice variants or allelic variants. The sequence of an exemplary human MYC nucleic acid is NG_{—}007161. An exemplary human MYC amino acid sequence is NP_{—}002458. An exemplary chimpanzee MYC nucleic acid sequence is NC_{—}006475.3 (126418056-126422420). An exemplary chimpanzee MYC amino acid sequence is NP_{—}001136266.1. An exemplary rhesus monkey MYC nucleic acid sequence is NC_{—}007865.1 (130351288-130353974). An exemplary rhesus monkey MYC amino acid sequence is NP_{—}001136345.1. An exemplary dog MYC nucleic acid sequence is NC_{—}006595.3 (25200772-25205309). An exemplary dog MYC amino acid sequence is NP_{—}001039539.1. An exemplary cow MYC nucleic acid sequence is AC_{—}000171.1 (13769242-13774438). An exemplary cow MYC amino acid sequence is NP_{—}001039539. An exemplary mouse MYC nucleic acid sequence is NC_{—}000081.6 (61985341-61990361). An exemplary mouse MYC amino acid sequence is NP_{—}034979.3. An exemplary rat MYC nucleic acid sequence is NC_{—}005106.3 (03157452-103162379). An exemplary rat MYC amino acid sequence is NP_{—}036735.2. An exemplary chicken MYC nucleic acid sequence is NC_{—}006089.3 (139318206-139320443). An exemplary chicken MYC amino acid sequence is NP_{—}001026123.1. An exemplary zebrafish MYC nucleic acid sequence is NC_{—}007135.5 (10214238-10216778). An exemplary zebrafish MYC amino acid sequence is NP_{—}571487.2. In some embodiments, MYC includes any MYC (or fragment or variant thereof) that supports cell (e.g. hematopoietic stem cell) proliferation and/or survival.

“Bcl-2” or “B-cell lymphoma 2” as used herein refers to a native Bcl-2 or Bcl-2 family member from any vertebrate source, including mammals such as primates (e.g., humans) and rodents (e.g., mice and rats), unless otherwise indicated. The terms encompass the genomic location, (e.g., 18q21.3 cytogenic band, chromosome 18:60790579-60987361, and/or GC18M060763), “full-length,” unprocessed Bcl-2 as well as any form of Bcl-2 that result from processing in the cell. The terms also encompass naturally occurring variants of Bcl-2, e.g., splice variants or allelic variants. The sequence of an exemplary human Bcl-2 nucleic acid is NG_{—}009361.1. An exemplary human Bcl-2 amino acid sequence is NP_{—}000624. An exemplary chimpanzee Bcl-2 nucleic acid sequence is NC_{—}006485.3 (59230514-59427579). An exemplary chimpanzee Bcl-2 amino acid sequence is XP_{—}0011455371. An exemplary dog nucleic acid sequence is NC_{—}006583.3 (13733849-13900653). An exemplary dog amino acid sequence is NP_{—}001002949.1. An exemplary cow nucleic acid sequence is AC_{—}000181.1 (61914152-62105526). An exemplary cow amino acid sequence is NP_{—}001159958.1. An exemplary mouse nucleic acid sequence is NC_{—}000067.6 (106538179-106714290). An exemplary mouse amino acid sequence is NP_{—}033871.2. An exemplary rat nucleic acid sequence is NC_{—}005112.3 (31758213-31919850). An exemplary rat amino acid sequence is NP_{—}058689.1. An exemplary chicken nucleic acid sequence is NC_{—}006089.3 (67926749-68013683). An exemplary chicken amino acid sequence is NP_{—}990670.1. In some embodiments, a Bcl-2 family member includes any Bcl-2 homologue (or fragment or variant thereof) that prevents apoptosis (e.g. apoptosis of a stem cell, optionally a hematopoietic stem cell or derivative thereof).

The term “tat” peptide as used herein refers to the trans-activating transcriptional activator (Tat) from Human Immunodeficiency Virus 1 (HIV-1). An exemplary tat peptide is a peptide with the amino acid sequence RKKRRQRRR. An exemplary tat peptide may be encoded by a nucleic acid with the sequence 5′-aggaagaagcggagacagcgacgaaga-3′. In some examples, tat peptides increase cellular uptake of macromolecules such as polypeptides.

The term “stem cells” as used herein refers to cells that are capable of dividing and renewing themselves for long periods, are unspecialized (undifferentiated), and can give rise to (differentiate into) specialized cell types (i.e., they are progenitor or precursor cells for a variety of different, specialized cell types). “Long-term”, when used in connection with stem cells, refers to the ability of stem cells to renew themselves by dividing into the same non-specialized cell type over long periods (e.g., many months, such as at least 3 months, to years) depending on the specific type of stem cell. “Stem cells” as used herein refers to cells that are capable of dividing and self-renewing to produce more cells over a period of time, and can be unspecialized (undifferentiated or pluripotent) or specialized (progenitor or precursor) cell types, for example, hematopoietic stem cells (HSCs). Hematopoietic stem cells can be obtained from any source, for example, bone marrow, cord blood, peripheral blood, mobilized bone marrow, or reprogrammed somatic cells. “Long-term” or “Lt,” when used in conjunction with stem cells, refers to the continued ability of stem cells to self-renew for an extended period of time, for example several months. The cell surface marker phenotype of lt-HSCs may vary by species, for example human lt-HSCs exhibit a cell surface marker phenotype of CD34⁺, Lin⁻, Flk-2⁻, c-kit⁺, while murine lt-HSCs can be identified by the cell surface marker phenotype CD34⁻, Flk-2⁻, c-kit⁺, Sca-1⁺.

“Conditionally immortalized long-term” or “ctlt” when used in conjunction with stem cells, refers to stem cells that are immortalized (capable of indefinite self-renew without differentiation under cytokine dependent conditions), but maintain the ability to become non-immortal and differentiate into other cell-type lineages under specific conditions. In some embodiments, conditionally immortalized stem cells are hematopoietic stem cells that have been conditionally immortalized by exposure to a MYC that promotes cell proliferation and/or survival, and a Bcl-2 family member that inhibits apoptosis. In some embodiments, exposure is through overexpression of genes encoding the MYC and the Bcl-2. In some embodiments the gene encoding MYC is inducible (e.g. through fusion with a hormone and/or drug regulatory region such as but not limited to MYC-ER). In some embodiments, the gene encoding Bcl-2 is inducible. In some embodiments, exposure is through protein transduction of the MYC and the Bcl-2 polypeptides. Such conditionally immortalized HSCs and methods of making are described in, for example, US Patent Application Publication No. 2010/0297763 A1, incorporated herein by reference in its entirety.

The term “immunodeficient” as used herein refers to an animal's impaired or otherwise not fully functioning immune system, for example an inability to produce a normal amount of B-cells, T-cells, NK-cells, etc. Immunodeficiency may be produced by, for example, but not limited to, mutations, irradiation, a chemical or pharmaceutical, or a virus. Examples of immunodeficient mice include but are not limited to NSG mice (NOD/SCID/γc^{-/-}; or NOD/scid IL2rγ^{null}), NOG mice (NOD/γc^{-/-} or NOD/scid/IL2rγ^{Trunc}), NOD mice (non-obese diabetic), SCID mice (severe combined immunodeficient mice), NOD/SCID mice, nude mice, BRG mice (BALB/c-Rag2^{null}/IL2rγ^{null}), Rag 1^{-/-} mice, Rag 1^{-/-}/γc^{-/-} mice, Rag 2^{-/-} mice, and Rag 2^{-/-/γc}^{-/-} mice. In some examples, mice that have been cross-bred with any of the above-referenced mice and have an immunocompromised background may be used for implanting HSCs as described herein. In some examples, the immune deficiency may be the result of a genetic defect in recombination, a genetically defective thymus, a defective T-cell receptor region, a NK cell defect, a Toll receptor defect, an Fc receptor defect, an immunoglobulin rearrangement defect, a defect in metabolism or any combination thereof. In some examples, mice are rendered immunedeficient by administration of an immunosuppressant, e.g. cyclosporin, NK-506, removal of the thymus, or radiation.

The term “immunocompetent” as used herein refers to an animal with a functioning immune system and otherwise not immunodeficient. An immunocompetent animal can include, for example, an otherwise immunodeficient animal with a reconstituted immune system. In one embodiment, an immunocompetent animal will be a wild-type mouse. In another embodiment, an immunocompetent animal will be a sublethally irradiated NSG mouse with a successfully transplanted bone marrow and exhibiting mature T-cells, B-cells, and NK-cells.

The terms “subject” and “patient” are used interchangeably herein to refer to a human and/or a non-human animal. In some embodiments, methods of treating other mammals, including, but not limited to, rodents, simians, felines, canines, equines, bovines, porcines, ovines, caprines, mammalian laboratory animals, mammalian farm animals, mammalian sport animals, and mammalian pets, are also provided.

The term “cohort” as used herein refers to a group of individuals with a common characteristic, such as a common statistical characteristic. In some embodiments, a cohort of xenochimaeric animals are animals which share common characteristic such as HSCs from a common heterologous animal.

The term “population” as used herein refers to a plurality of individuals. For example, a population of xenochimaeric mice may encompass a plurality of mice in which heterologous HSCs have been introduced. In some embodiments, the heterologous HSCs are from the one individual (e.g. a human or a non-human animal) or from more than one individual. In some embodiments, the more than one individual share a common trait; e.g. HLA type.

The term “cancer” refers to a proliferative disorder associated with uncontrolled cell proliferation, unrestrained cell growth, and decreased cell death via apoptosis.

The term “tumor” is used herein to refer to a group of cells that exhibit abnormally high levels of proliferation and growth. A tumor may be benign, pre-malignant, or malignant; malignant tumor cells are cancerous. Tumor cells may be solid tumor cells or leukemic tumor cells. The term “tumor” as used herein also refers to a portion of a tumor; for example a sample of a tumor. A sample of a tumor may be divided into smaller portions and engrafted in a plurality of xenochimaeric non-human animal hosts (e.g. mice) to generate a population of xenochimaeric non-human animals (e.g. mice). The term “tumor growth” is used herein to refer to proliferation or growth by a cell or cells that comprise a tumor that leads to a corresponding increase in the size of the tumor.

As used herein, “treatment” is an approach for obtaining beneficial or desired clinical results. For purposes of this invention, beneficial or desired clinical results include, but are not limited to, any one or more of: alleviation of one or more symptoms, diminishment of extent of disease, preventing or delaying spread (e.g., metastasis, for example metastasis to the lung or to the lymph node) of disease, preventing or delaying recurrence of disease, delay or slowing of disease progression, amelioration of the disease state, and remission (whether partial or total). Also encompassed by “treatment” is a reduction of pathological consequence of a proliferative disease. The methods of the invention contemplate any one or more of these aspects of treatment.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

An “effective amount” of an agent refers to an amount effective, at dosages and for periods of time necessary, to achieve the desired therapeutic or prophylactic result.

A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

To “reduce” or “inhibit” is to decrease or reduce an activity, function, and/or amount as compared to a reference. In certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater. Reduce or inhibit can refer to the symptoms of the disorder being treated, the presence or size of metastases, the size of the primary tumor, or the size or number of the blood vessels in angiogenic disorders.

The term “test agent” refers to a candidate agent that is evaluated using the xenochimaeric animal of the invention to determine the efficacy of the agent for treatment of a disease or disorder; e.g. cancer. The test agent may be used in the xenochimaeric animal to determine the efficacy of the test agent to treat the disease or disorder of the xenochimaeric animal. For example, a xenochimaeric animal model for a particular cancer can be used to test known or potential agents to for efficacy in treatment of the cancer. In some embodiments, the test agent is a known therapeutic agent. In some embodiments, the therapeutic activity of the test agent is unknown. For example, the test agent may be a potential new therapeutic treatment for the disease of the xenochimaeric model. In some embodiments, the test agent is a test anticancer agent. Examples of anti-cancer agents include, but are limited to, e.g., chemotherapeutic agents, growth inhibitory agents, cytotoxic agents, radiation, agents used in radiation therapy, anti-angiogenesis agents, apoptotic agents, anti-tubulin agents, and other agents to treat cancer. In some embodiments the test agent is a treatment paradigm; for example, but not limited to a dosing regimen, a combination of therapies, and the like.

The term “pharmaceutical formulation” refers to a preparation which is in such form as to permit the biological activity of the active ingredient to be effective, and which contains no additional components which are unacceptably toxic to a subject to which the formulation would be administered. Such formulations may be sterile.

A “sterile” formulation is aseptic or free from all living microorganisms and their spores.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

As used herein, “in conjunction with” refers to administration of one treatment modality in addition to another treatment modality. As such, “in conjunction with” refers to administration of one treatment modality before, during or after administration of the other treatment modality to the individual.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise.

It is understood that aspects and variations of the invention described herein include “consisting” and/or “consisting essentially of” aspects and variations.

### II. METHODS OF GENERATING XENOCHIMAERIC NON-HUMAN ANIMALS (E.G. MICE)

The invention provides methods of generating xenochimaeric animals (e.g. mice) comprising HSCs and tumors from one or more heterologous animals. In some embodiments, the invention provides methods of generating xenochimaeric mice comprising HSCs and all or a portion of a tumor from one or more heterologous animals. In some embodiments, the HSCs and the tumor, or portion thereof, are from the same species of heterologous animal. In some embodiments, the HSCs are human HSCs and the tumor is a human tumor, or portion thereof. In some embodiments, the HSCs and the tumor, or portion thereof, are from the same individual; for example, the HSCs and the tumor are from the same human cancer patient.

### A. Conditionally Immortalized Long Term Stem Cells

The invention provides methods to conditionally immortalize HSCs to produce long term HSCs prior to introduction to xenochimaeric mice. Conditionally immortalizing HSCs prior to implantation provides multiple benefits, including but not limited to, enhanced engraftment optionally requiring fewer cells, and optionally beginning with a smaller original blood sample. The process of conditional immortalization also allows for preferential expansion of the number HSCs. Further, such expansion may be performed on the blood sample without the need for sorting and isolation of a particular starting HSC lineage. As a result of one or more of these advantages, limited patient samples are sufficient to create cohorts of xenochimaeric animals to allow for testing of a variety of treatments (e.g. patient-specific treatments), and to allow comparability and reproducibility of results over time. In addition, since the conditionally immortalized HSCs can be propagated through one or more or multiple generations, the resulting xenochimaeric animal system has enhanced comparability and reproducibility across cohorts and/or time for use as a drug screening system.

Methods to conditionally immortalize HSCs are provided by US Patent Application Publication No. 2010/0297763 A1, incorporated herein by reference in its entirety. In one embodiment, the method includes the following steps: (a) obtaining an expanded population of adult stem cells; (b) culturing the HSCs in the presence of a protooncogene product that promotes cell survival and/or proliferation; (c) culturing the HSCs in the presence of a polypeptide that inhibits apoptosis of the cell; and (d) expanding the HSCs in the presence of a combination of stem cell growth factors under conditions whereby the protooncogene product is active. In some embodiments, the protooncogene product and/or the polypeptide that inhibits apoptosis is removed or inactivated prior to implantation of the HSCs in a xenochimaeric mouse.

As used herein, the phrase “conditionally immortalized” refers to cells that are immortalized (e.g., capable of indefinite growth without differentiation in a cytokine dependent fashion, while maintaining their ability and potential to differentiate into a number of different lineages under the appropriate conditions) in a reversible manner, such that the cells are immortalized under a specific set of conditions, and when the conditions are removed or changed (or other conditions added), the cells are no longer immortalized and may differentiate into other cell types. In some embodiments, the cells are subject to a protooncogene product that promotes cell survival and/or proliferation. In some embodiments, the cells are subject to a polypeptide that inhibits apoptosis of the cell. In some embodiments, the cells are subject to a protooncogene product that promotes cell survival and/or proliferation and to a polypeptide that inhibits apoptosis of the cell. In some embodiments, the protooncogene product is a Myc family polypeptide; e.g. c-Myc, Myc-ER, n-Myc, L-Myc, etc. In some embodiments, the polypeptide that inhibits apoptosis is a Bcl-2 family polypeptide; e.g. Bcl-2, Bcl-xL, Bcl-w, etc.

As used herein, “stem cells” refers to the term as it is generally understood in the art. For example, stem cells, regardless of their source, are cells that are capable of dividing and renewing themselves for long periods, are unspecialized (undifferentiated), and can give rise to (differentiate into) specialized cell types (i.e., they are progenitor or precursor cells for a variety of different, specialized cell types). “Long-term”, when used in connection with stem cells, refers to the ability of stem cells to renew themselves by dividing into the same non-specialized cell type over long periods (e.g., many months, such as at least 3 months, to years) depending on the specific type of stem cell. Phenotypic characteristics of various long-term stem cells from different animal species, such as long-term hematopoietic stem cells (lt-HSCs) are known in the art. For example, murine lt-HSCs can be identified by the presence of one or more of the following cell surface marker phenotypes: c-kit⁺, Sca-1⁺, CD34⁻, flk2⁻. Human lt-HSCs may be identified by the presence of one or more of the following cell surface marker phenotypes: CD34⁺, CD38^{lo}, CD150⁺, CD48^{lo}, lin⁻. Adult stem cells include stem cells that can be obtained from any non-embryonic tissue or source, and typically generate the cell types of the tissue in which they reside. The term “adult stem cell” may be used interchangeably with the term “somatic stem cell”. Embryonic stem cells are stem cells obtained from any embryonic tissue or source. Hematopoietic stem cells give rise to all of the types of blood cells, including but not limited to, red blood cells (erythrocytes), B lymphocytes, T lymphocytes, natural killer cells, neutrophils, basophils, eosinophils, monocytes, macrophages, and platelets.

HSCs in the present invention can be obtained from any source. For example, HSCs can be obtained from bone marrow or cord blood. In another embodiment, stem cells are derived from peripheral blood, or derived from any pluripotent stem cells, including embryonic stem cells. For example, HSCs can be obtained from a donor treated with an agent that enriches HSCs and encourages such cells to expand without differentiation. In one embodiment of the invention, HSCs can be obtained from the peripheral blood of a human patient treated with granulocyte-colony stimulating factor (G-CSF). In another embodiment of the invention, HSCs can be obtained from the peripheral blood of a human patient treated with granulocyte-colony stimulating factor (G-CSF) undergoing a chemotherapy regime. Expanding HSCs (lt-HSCs) can be further isolated by any manner known in the art. For example, conjugated antibodies against HSC cell surface marker proteins can be used to isolate the cells. In one embodiment, magnetic beads coated with human CD34 antibody can be mixed with human whole cord blood or G-CSF mobilized human peripheral blood to isolate human lt-HSCs.

Methods to grow lt-HSCs have been described in US Patent Application Publication Nos. 2007/0116691 and 2010/0297763, and European Patent No. 1942739; the contents of each are herein incorporated in their entireties.

In some embodiments of the invention, HSCs are conditionally immortalized by culturing the cells in the presence of Myc and/or Bcl-2. In some embodiments of the invention, HSCs cultured in the presence of Myc and/or Bcl-2 polypeptides. In some embodiments, the Myc and/or Bcl-2 polypeptides are added exogenously to the culture of stem cells. In some embodiments, the Myc and/or Bcl-2 polypeptides are fused to one or more peptides to enhance cellular uptake of the Myc and/or Bcl-2 polypeptides. In some embodiments, the Myc and/or Bcl-2 polypeptides are fused to a Tat polypeptide derived from human immunodeficiency virus (HIV). In some embodiments, the Myc and/or Bcl-2 polypeptides are provided to the HSCs culture by introducing nucleic acids expressing Myc and/or Bcl-2 to the HSCs. In some embodiments, the nucleic acids encoding Myc and/or Bcl-2 are introduced to the HSCs using one or more viral vectors, optionally one or more integrating viral vectors. Examples of viral vectors include, but are not limited to retroviral vectors, lentivirus vectors, parvovirus vectors such as adeno-associated viral vectors, vaccinia virus vectors, coronavirus vectors, calicivirus vectors, papilloma virus vectors, flavivirus vectors, orthomixovirus vectors, togavirus vectors, picornavirus vectors, adenoviral vectors, and herpesvirus vectors. In some embodiments, nucleic acids encoding Myc and/or Bcl-2 are transfected into the HSCs using direct electroporation. In some embodiments, nucleic acid encoding Myc and/or Bcl-2 are introduced to cells that are co-cultured with the HSCs; for example, feeder cells.

In some embodiments of the invention, human cord blood cells are used as a source to produce ctlt-HSCs. In an exemplary method, red blood cells are lysed by incubating cord blood cells in a hypotonic lysis buffer. The remaining cells are cultured in Stem line media (Stem Cell Technologies) supplemented with IL-3, IL-6 and SCF as well as 20 mg/mL of each Tat-MYC and Tat-Bcl-2. Cells are incubated in 24 well plates in 1 mL of medium, with a starting density of 2×10⁶ cells per well. The medium is replaced every two days. After 14 days, FACS data is collected to observe an increase in percentage of CD38⁺, CD34⁺ HSCs. The total CD34⁺ HSCs in the culture may be regularly monitored, with regular increase in the total number of lt-HSCs demonstrating the formation of ctlt-HSCs. The total number of human lt-HSCs may increase steadily in the cultures over the period in which they are analyzed. The HSCs obtained from this culture on day 28 may be used to generate xenochimaeric mice.

B. Formation of Xenochimaeric Animals (e.g. Mice)

The invention provides methods to generate xenochimaeric animal (e.g., mice) by populating animals with HSCs from a heterologous animal followed by implantation of tumor. In some embodiments, the HSCs are ctlt-HSC prepared as described above. In some embodiments of the invention, the recipient animals (e.g. mice) are immunodeficient animals (e.g. mice). In some embodiments of the invention, immunodeficient mice are used for the generation of xenochimaeric mice. In some embodiments, mice are deficient in their immune system as a result of a genetic defect. In some embodiments, the mice lack at least one or more or all of T cells, B cells, or NK cells. In some embodiments the T cells, B cells, or NK cells are unable to undergo or to complete maturation. In some embodiments, the genetic defect is a naturally occurring genetic defect. In some embodiments, the genetic defect is induced. Examples of immunodeficient mice include but are not limited to NSG mice (NOD/SCID/γc^{-/-}; or NOD/scid IL2rγ^{null}), NOG mice (NOD/γc^{-/-} or NOD/scid/IL2rγ^{Trunc}), NOD mice (non-obese diabetic), SCID mice (severe combined immunodeficient mice), NOD/SCID mice, nude mice, BRG mice (BALB/c-Rag2^{null}/IL2rγ^{null}), Rag 1^{-/-} mice, Rag 1^{-/-}/γc^{-/-} mice, Rag 2^{-/-} mice, and Rag 2^{-/-}/γc^{-/-} mice. In some embodiments, mice that have been cross-bred with any of the above-referenced mice and have an immunocompromised background may be used for implanting HSCs as described herein. In some embodiments, the immune deficiency may be the result of a genetic defect in recombination, a genetically defective thymus, a defective T-cell receptor region, a NK cell defect, a Toll receptor defect, an Fc receptor defect, an immunoglobulin rearrangement defect, a defect in metabolism or any combination thereof. In some embodiments, mice are rendered immunedeficient by administration of an immunosuppressant, e.g. cyclosporin, NK-506, removal of the thymus, or radiation.

In some embodiments, the non-human animal (e.g. mice) are sublethally irradiated prior to the introduction of HSCs (e.g. ctlt-HSCs) from a heterologous animal. In some embodiments, immunodeficient mice are sublethally irradiated prior to the introduction of HSCs from a heterologous animal. In some embodiments, immunodeficient mice are sublethally irradiated prior to introduction of human HSCs. In some embodiments, the mice are irradiated with γ radiation at an amount of about 100 rads, about 200 rads, about 300 rads, about 400 rads, or about 500 rads prior to introduction of heterologous HSCs. In some embodiments, mice are irradiated with γ radiation at a dose of about 10 cGy/g body weight, about 11 cGy/g body weight, or about 12 cGy/g body weight. In some embodiments, the amount of sublethal irradiation used is determined based on the ability of the transplanted cells to engraft the bone marrow of the non-human animal host. In some embodiments, the γ radiation is ¹³⁷Cs γ radiation. In some embodiments, the radiation is an X-ray. In some embodiments, the mice are irradiated with X radiation at an amount of about 100 rads, about 200 rads, about 300 rads, about 400 rads, or about 500 rads prior to introduction of heterologous HSCs. In some embodiments, mice are irradiated with X radiation at a dose of about 10 cGy/g body weight, about 11 cGy/g body weight, or about 12 cGy/g body weight.

In some embodiments of the invention, heterologous HSCs (e.g. ctlt-HSCs) are introduced to immunodeficient animals (e.g., mice) to generate xenochimaeric animals (e.g. mice). In some embodiments, heterologous HSCs are introduced to sublethally irradiated mice to generate xenochimaeric mice. In some embodiments, heterologous HSCs are introduced to sublethally irradiated immunodeficient mice to generate xenochimaeric mice. In some embodiments, the heterologous HSCs are human HSCs. Methods to introduce heterologous HSCs to mice include but are not limited to intravenous injection, intraarterial injection, subcutaneous injection, intraperitoneal injection, and intramuscular injection.

In some embodiments, the HSCs are introduced to the immunodeficient animals (e.g. mice) about 8 weeks to about 10 weeks prior to introduction of the tumor to the mice.

In some embodiments, blood samples, e.g., peripheral blood samples, from xenochimaeric animals are analyzed for the presence of heterologous hematopoietic-derived cells. For example, blood samples may be analyzed every week, every two weeks, every three weeks, or every four weeks or more for the presence of heterologous hematopoietic-derived cells. In some embodiments, human HSCs are introduced to immunodeficient mice to generate xenochimaeric mice. The presence of heterologous hematopoietic-derived cells in the peripheral blood is an indicator of successful engraftment in the non-human animal. Blood samples are analyzed for human CD45⁺ and human CD3⁺ cells. The presence of CD45⁺/CD3⁺ cells indicates engraftment of human HSCs in the xenochimaeric mice. In some embodiments, xenochimaeric mice are fully constructed about 10 to about 12 weeks after the ctlt-HSCs are injected into mice.

C. Implantation of a Heterologous Tumor into Xenochimaeric Animals (e.g. Mice)

The invention provides methods of introducing one or more heterologous tumors to xenochimaeric animals (e.g. mice). In some embodiments, the heterogeneous tumor is a portion of a heterogeneous tumor. In some embodiments, a heterologous tumor is introduced to a xenochimaeric mouse wherein the heterologous tumor is from the same species as the heterologous HSCs used to generate the xenochimaeric mouse. In some embodiments, a heterologous tumor is introduced to a xenochimaeric mouse wherein the heterologous tumor is from the same individual as the heterologous HSCs used to generate a xenochimaeric mouse. In some embodiments, a human tumor is introduced to a xenochimaeric mouse wherein the xenochimaeric mouse was generated using human HSCs. In some embodiments, a tumor from a cancer patient is introduced to a xenochimaeric mouse wherein the xenochimaeric mouse was generated using HSCs from the cancer patient. In some embodiments, a human tumor is introduced to an autologous xenochimaeric mouse.

In some embodiments, the tumor is from a non-human animal. Examples of non-human animals include, but are not limited to, domesticated animals (dogs, cats, rabbits, horses and the like), farm animals (cows, pigs, horses and the like), human companion animals, zoo animals, wild animals, laboratory animals (rats, mice, hamsters, guinea pigs, monkeys, apes, and the like). In some embodiments, the non-human animal is a canine (e.g. dog) or a feline (e.g. cat).

In some embodiments of the invention, the heterologous tumor, or portion thereof, is a malignant tumor. In some embodiments of the invention, the heterologous tumor, or portion thereof, is a benign tumor. In some cases, benign tumors may represent significant clinical problems and/or may behave like malignant tumors. Such benign tumors include but are not limited to pituitary adenomas, neuromas, neurofibromas, and/or meningiomas. In some embodiments of the invention, the heterologous tumor is a solid tumor. In some embodiments, the tumor is a portion of a tumor. Examples of solid tumors include, but are not limited to, head and neck tumors, brain tumors, eye tumors, thyroid tumors, adrenal tumors, salivary gland tumors, esophageal tumors, gastric tumors, intestinal tumors, colon tumors, lung tumors, breast tumors, liver tumors, pancreatic tumors, kidney tumors, bladder tumors, prostate tumors, muscular tumors, osseous tumors, skin tumors, myeloblastomas, lymphomas, non-Hodgkins lymphomas and stromal/sarcoma tumors. In some embodiments, the tumor, or portion thereof, is a primary tumor. In some embodiments, the tumor is metastases. In some embodiments of the invention, the tumor is a human tumor. In some embodiments, the tumor, or portion thereof, is derived from a cancer patient undergoing anti-cancer therapy; e.g. chemotherapy or radiation therapy. In some embodiments, the tumor, or portion thereof, is derived from a patient who has not undergone anti-cancer therapy.

In some embodiments of the invention, the tumor is a population of blood cancer cells. In some embodiments of the invention, blood cancer cells are introduced into the xenochimaeric mice. In some embodiments, human blood cancer cells are introduced into the xenochimaeric mice. Examples of blood cancer cells include acute lymphoblastic leukemia (ALL) cells, acute myelogenous leukemia (AML) cells, chronic lymphocytic leukemia (CLL) cells, and chronic myelogenous leukemia (CML) cells. In some embodiments, the blood cancer cell is derived from a cancer patient undergoing anti-cancer therapy; e.g. chemotherapy or radiation therapy. In some embodiments, the blood cancer cell is derived from a patient who has not undergone anti-cancer therapy. In some embodiments, the cancer patient is a human. In some embodiments, the cancer patient is a non-human animal; e.g. a pet such as a dog or cat.

In some embodiments of the invention, a heterologous tumor, or portion thereof, is engrafted into a xenochimaeric mouse. In some embodiments, the heterologous tumor, or portion thereof, is implanted subcutaneously in a xenochimaeric mouse. In some embodiments, the tumor, or portion thereof, is implanted subcutaneously in the flank of a xenochimaeric mouse. In some embodiments, a human tumor, or portion thereof, is implanted subcutaneously in the flank of a xenochimaeric mouse wherein the xenochimaeric mouse was generated using human HSCs. In some embodiments, a tumor, or portion thereof, from a human cancer patient is implanted subcutaneously in the flank of a xenochimaeric mouse wherein the xenochimaeric mouse was generated using human HSCs from the same human cancer patient. In some embodiments, the HSCs are conditionally immortalized HSCs (e.g. ctlt-HSCs, or created through the use of Tat-Myc and Tat-Bcl-2, as described herein).

In some embodiments, the tumor, or portion thereof, is implanted orthotopically. In some embodiments, the tumor, or portion thereof, is implanted orthotopically to the corresponding site of a xenochimaeric mouse from where the tumor was derived. For example, a liver tumor may be implanted in the liver of a xenochimaeric mouse or a brain tumor may be implanted in the brain of a xenochimaeric mouse. In some embodiments, more than one tumor of the same origin, or portion thereof, is implanted in the mouse. For instance, portions from the same tumors may be implanted both orthotopically and heterotopically to assess a difference in effect related to the implantation site. Or one tumor may be implanted in an area exposed to radiation (i.e., in the flank), whereas another portion is implanted in an area away from the radiation field (i.e., in the axilla), to explore the difference in the effectiveness of direct radiation versus radiation that is not directly on a tumor (e.g. the indirect effect of radiation on one tumor as compared with the direct effect of radiation on another tumor in the same animal host).

In some embodiments, portions from different tumors may be implanted on the same xenochimaeric mouse. For instance this may be used to explore the differential effect of a therapy, cytokine, or immune therapy depending on the characteristics of the tumor, or depending of the phenotype of the HSCs used to generate the xenochimaric mice. An example would be implanting un-matched HSCs from a given HLA type with two tumors, one with identical HLA and the other with a different HLA, and explore how this impacts efficacy of the said intervention.

In some embodiments of the invention, the tumor, or portion thereof, is implanted in a xenochimaeric mouse at a site that differs from the site from where the tumor was derived. For example, a brain tumor may be implanted into the liver of a xenochimaeric mouse or a liver tumor may be implanted into the kidney of a xenochimaeric mouse. In some embodiments, a human tumor, or portion thereof, is implanted orthotopically in a xenochimaeric mouse wherein the xenochimaeric mouse was generated using human HSCs. In some embodiments, the HSCs are conditionally immortalized HSCs (e.g. ctlt-HSCs, or created through the use of Tat-Myc and Tat-Bcl-2, for example, as described above and/or herein).

In some embodiments, a tumor, or portion thereof, from a human cancer patient is implanted orthotopically in a xenochimaeric mouse wherein the xenochimaeric mouse was generated using human HSCs from the same human cancer patient. In some embodiments, a tumor, or portion thereof, from a non-human cancer patient is implanted orthotopically in a xenochimaeric mouse wherein the xenochimaeric mouse was generated using HSCs from the same non-human cancer patient.

In some embodiments of the invention, the tumor, or portion thereof, is implanted in a xenochimaeric mouse by a hematogenous route. In some embodiments, the tumor is introduced to a xenochimaeric mouse intravenously or intraarterially. In some embodiments, a human tumor, or portion thereof, is implanted via a hematogenic route in a xenochimaeric mouse wherein the xenochimaeric mouse was generated using human HSCs. In some embodiments, a tumor, or portion thereof, from a human cancer patient is implanted via a hematogenic route in a xenochimaeric mouse wherein the xenochimaeric mouse was generated using human HSCs from the same human cancer patient. In some embodiments, a non-human tumor, or portion thereof, is implanted via a hematogenic route in a xenochimaeric mouse wherein the xenochimaeric mouse was generated using HSCs from a non-human animal. In some embodiments, a tumor, or portion thereof, from a non-human cancer patient is implanted via a hematogenic route in a xenochimaeric mouse wherein the xenochimaeric mouse was generated using HSCs from the same non-human cancer patient.

In some embodiments of the inventions, the tumor, or portion thereof, is removed from a heterologous animal and implanted directly into a xenochimaeric mouse. In some embodiments a tumor is removed from a heterologous animal and cut into small pieces (e.g. about 20-25 mm³) and implanted into one or more xenochimaeric mice. In some embodiments, the tumor, or portion thereof, is washed, e.g. in a saline solution, prior to implantation in a xenochimaeric mice. In some embodiments, the tumor, or portion thereof, is incubated in a culture medium prior to implantation in a xenochimaeric mouse. In some embodiments, the tumor, or portion thereof, is incubated for one or two days prior to implantation in a xenochimaeric mouse. In some embodiments, cells of the tumor are not allowed to replicate prior to implantation in a xenochimaeric mouse. In some embodiments, the solid tumor is not dissociated prior to implantation; e.g. the solid tumor is not dissociated to portions of ten cells or less prior to implantation in a xenochimaeric mouse. In some embodiments, the tumor is not a tumor cell line. Implanting non-dissociated primary tumors from the patient is important since passage of such tumors on non-humanized models leads to loss of stroma after 1-3 passages and complete substitution by stroma of mouse origin. The xenochimaeric model system described herein has the potential to preserve original non-cancerous components within the tumor (as well as to enable re-establishment of heterologous stromal components). Such components include, but are not limited to, macrophage, B cell, T cell, NK cells, fibroblasts, myofibroblasts, endothelial cells, blood vessels, and/or lymph vessels. Maintaining (or re-establishing) the original stroma is relevant to discover and develop new anticancer targets relevant to the tumor-stroma interaction, and for patient treatment assessment in order to develop individualized anticancer therapies since other models do not account for that lost stroma.

In some embodiments, the introduction of the HSCs and the tumor result in formation of stroma corresponding to the heterologous animal. In some embodiments, the introduction of the HSCs and the tumor result in reversion of one or more of tumor phenotype or tumor genotype towards the phenotype of genotype of the tumor initially isolated from the heterologous animal.

In some embodiments, the invention provides methods to monitor the growth of a tumor after engraftment. In some embodiments, cancer stem cells (CSCs) for a xenochimaeric mouse are analyzed. In some embodiments, cancer stem cells (CSCs) for a xenochimaeric mouse comprising human HSCs and a human tumor are analyzed; for example, by FACS for CD44⁺, CD24⁺ cells, and/or ALDH⁺ cells. In some embodiments, the size of the engrafted tumor is monitored.

The invention provides methods to demonstrate progenitor homing from the heterologous bone marrow to the tumor. For example, the stroma of the engrafted human tumors may be analyzed by measuring the percentage of human (or corresponding other non-mouse engrafted cells) CD151⁺, CD31⁺, 1y1⁺, CD 45⁺, CD3⁺, CD19⁺, CD68⁺, CD4⁺, SMA⁺, and/or CD57⁺ cells in the tumor. In some embodiments, tumors are analyzed at time periods following tumor engraftment; for example, one month and three months after tumor engraftment. Cells that may be assessed as part of the stroma include, but are not limited to, macrophage, B cell, T cell, NK cells, fibroblasts, myofibroblasts, endothelial cells, blood vessels, and/or lymph vessels.

Maintaining original stroma can be relevant to discover and develop new anticancer targets relevant to the tumor-stroma interaction, and it can be relevant for patient treatment assessment in order to develop individualized anticancer therapies since other models cannot account for that lost stroma. Implanting dissociated components separately is a valid strategy for biologic understanding of each stroma component's contribution and is a powerful tool for discovery of anticancer targets (for instance, in defining the relevance of human fibroblast WNT pathway signaling in establishing a tumor it is relevant to implant in xenochimaeric hosts tumor and fibroblasts cells only). Implanting entire portions of tumors and maintaining their heterogeneity by subsequent passage on the xenochimaeric host is relevant for assessing patient treatments and drug screening since for instance maintaining cancer cells, fibroblasts and immune cells will be relevant to test a combination of cytotoxic drug plus a WNT inhibitor plus an immune modulator. The simultaneous presence and interaction of said components lends uniqueness to the development and discovery properties of Xenochimaeric mice.

In some embodiments, stromal elements may be visualized using a differential fluorescence in situ hybridization (FISH) DNA staining assay of the engrafted tumors. For example, by staining human DNA as red and mouse DNA as green using human and mouse Cot-1 DNA immunofluorescence probes, a semi-quantitative assessment of the ratio of human-to-mouse cells may verify more human stromal elements and less mouse stromal elements in xenochimaeric mice compared to a control immunodeficient mouse comprising a human tumor.

In some embodiments, heterologous tumors may be analyzed for heterologous stromal cells by DNA fingerprinting short tandem repeat (STR) analysis. For example, the origin of human CD151⁺, CD31⁺, 1y1⁺, CD 45⁺, CD3⁺, CD19⁺, CD68⁺, CD4⁺, SMA⁺, and/or CD57⁺ cells in xenochimaeric mice comprising human HSCs and human tumors may be determined by using primer sets for the human thyroid peroxidase (TPDX) and von Willebrand Factor type A (vWA) loci. The STR fingerprint obtained by this analysis may be compared to that obtained by amplification of previously isolated mouse genomic DNA and genomic DNA from the patient tumor originally grafted into the xenochimaeric mouse. A difference in PCR banding patterns observed from CD 151+ cell compared to the banding patterns of the patient tumor may indicate that stromal cells originated in the xenochimaeric mouse humanized bone marrow.

In one example, xenochimaeric mice may be generated from adult peripheral blood. The progeny of HSCs isolated from adult peripheral blood are present in the xenochimaeric mice. Flow cytometry can be used to show that no human CD45+/CD151+ cells are present in the bone marrow, spleen, blood or tumor of a control nude mouse into whose rear flank, 50,000 tumor cells are injected whereas human CD45+/CD151+ cells can be found in the bone marrow, spleen, peripheral blood and tumor of xenochimaeric mice. Human CD45+/CD151+ cells can also be found in the bone marrow, spleen, peripheral blood of xenochimaeric mice generated from the peripheral blood of a cancer patient given G-CSF while undergoing chemotherapy. Since the treatment effectively cures the patient, no tumor exists to implant in these xenochimaeric mice.

D. Populations of Xenochimaeric Animals (e.g. Mice)

As described above and herein, the invention provides methods to generate a population of xenochimaeric animals (e.g. mice). In some embodiments, heterologous ctlt-HSCs, prepared as described above and herein, are introduced to two or more immunodeficient mice, as described. In some embodiments, portions of a heterologous tumor are engrafted into the population of xenochimaeric mice comprising HSCs from a heterologous animal. In some embodiments, the portions of a tumor are derived from the same individual from which the HSC cells were derived to generate the population of xenochimaeric mice. In some embodiments, the HSCs are conditionally immortalized HSCs (e.g. ctlt-HSCs, or created through the use of Tat-Myc and Tat-Bcl-2, for example, as described above and/or herein). In some embodiments, portions of a human tumor are engrafted into a population of xenochimaeric mice comprising human HSCs. In some embodiments, portions of a tumor from a human cancer patient are engrafted into a population of xenochimaeric mice comprising HSCs from the same cancer patient. In some embodiments, portions of a non-human tumor are engrafted into a population of xenochimaeric mice comprising non-human HSCs. In some embodiments, portions of a tumor from a non-human cancer patient are engrafted into a population of xenochimaeric mice comprising HSCs from the same cancer patient.

In some embodiments, the populations of xenochimaeric animals (e.g. mice) are generated using conditionally immortalized HSCs. In some embodiments, the cells are subject to a protooncogene product that promotes cell survival and/or proliferation and to a polypeptide that inhibits apoptosis of the cell. In some embodiments, the protooncogene product is a Myc family polypeptide. In some embodiments, the polypeptide that inhibits apoptosis is a Bcl-2 family polypeptide.

In some embodiments, a population of xenochimaeric animals (e.g. mice) comprising heterologous HSCs and heterologous tumors wherein the heterologous HSCs and heterologous tumor is from the same individual (e.g. a human cancer patient or a non-human cancer patient). In some embodiments, a population of xenochimaeric animals (e.g. mice) comprising heterologous HSCs and heterologous tumors wherein the heterologous HSCs and heterologous tumor is from the different individuals. For example, the heterologous HSCs may be from a human that does not have cancer and the heterologous tumor is from a human cancer patient. In some embodiments, the heterologous HSCs and heterologous tumor from different individuals is matched in one or more characteristics. For example, the heterologous HSCs and tumor may be matched for one or more cell surface markers.

Cells of individual vertebrates may be identified by their major histocompatibility complex (MHC) cell surface antigen presenting proteins. In humans, the MHC in humans is known as the human leukocyte antigen (HLA) system. The major HLAs corresponding to MHC class I include HLA-A, HLA-B, and HLA-C. Minor MHC class I HLAs include HLA-E, HLA-F and HLA-G. HLAs corresponding to MHC class II include HLA-DP, HLA-DM, HLA-DOA, HLA-DOB, HLA-DQ, and HLA-DR. Each type comprises hundreds of subtype (e.g. HLA-B27), which are further subdivided in hundreds of sub-subtypes (e.g. HLA-B*2705). People will usually have 2 types of HLA-A, 2 of HLA-B, and 2 of HLA-C as well as 1 or 2 other types. HLA types are encoded in the HLA gene on chromosome 6 are hereditary. HLA types can be used in determining a match for transplantation similar to the ABO blood type. In some embodiments of the invention, the HLA type of the HSCs (e.g. ctlt-HSCs) are the same as the HLA type of the tumor. In some embodiments, the HLA type of the HSCs (e.g. ctlt-HSCs) are different than the HLA type of the tumor. In some embodiments, one or more HLA markers of the HSCs (e.g. ctlt-HSCs) is different from the HLA markers of the tumor.

In some embodiments of the invention, the heterologous tumor, or portion thereof, is a malignant tumor. In some embodiments of the invention, the heterologous tumor, or portion thereof, is a benign tumor. In some cases, benign tumors may represent significant clinical problems and/or may behave like malignant tumors. Such benign tumors include but are not limited to pituitary adenomas, neuromas, neurofibromas, and/or meningiomas. In some embodiments of the invention, the heterologous tumor is a solid tumor. In some embodiments, the tumor is a portion of a tumor. Examples of solid tumors include, but are not limited to, head and neck tumors, brain tumors, eye tumors, thyroid tumors, adrenal tumors, salivary gland tumors, esophageal tumors, gastric tumors, intestinal tumors, colon tumors, lung tumors, breast tumors, liver tumors, pancreatic tumors, kidney tumors, bladder tumors, prostate tumors, muscular tumors, osseous tumors, skin tumors, myeloblastomas, lymphomas, non-Hodgkins lymphomas and stromal/sarcoma tumors. In some embodiments, the tumor, or portion thereof, is a primary tumor. In some embodiments, the tumor is metastases. In some embodiments of the invention, the tumor is a human tumor. In some embodiments, the tumor, or portion thereof, is derived from a cancer patient undergoing anti-cancer therapy; e.g. chemotherapy or radiation therapy. In some embodiments, the tumor, or portion thereof, is derived from a patient who has not undergone anti-cancer therapy.

In some embodiments, the heterologous HSCs provided to the non-human animals (e.g. mice) of the populations of xenochimaeric animals include genetic modifications; for example definitive, transient or inducible. In some embodiments, the genetic modifications modify, suppress or enhance the expression of biologic molecules including, but not limited to, DNA, RNA, miRNA or protein following engraftment into the non-human animal. In some embodiments, the genetic modifications inhibit the expression or activation of genes that are relevant in the stroma to tumor interaction. In some embodiments, the genetic modifications include the insertion of vectors carrying short hairpin RNA (shRNA) inhibiting one or more of WNT7A, WNT4, WNT10A, WNT3A, WNT7B, WNT6, WNT16, WNT11, WNT9A, WNT5B, CSNK1E, AXIN1, DVL1, TCF3, MYC, JUN, MMP9, MMP10, MMP11, MMP12, MMP15, MMP17, MMP19, CLDN7, CLDN4, CLDN14, CLDN1, CLDN22, CLDN15, SNAIL TWIST1, or VIM. These genetic modifications may be utilized in models including but not limited to drug development, assay development, and biology development.

The conditional immortalization process allows the creation of cohorts/populations of mice starting from very few original HSCs. The ability to establish conditionally immortalized HSCs from limited amounts of blood permits establishing a conditionally immortalized HSC strain from peripheral blood or minimally invasive bone marrow biopsy from a patient (this is relevant since not all patients have the ability to allow a full apheresis or multiple HSC extractions, and since a less invasive blood draw is appropriate to direct HSC stabilization from a given donor/subject databank if a particular phenotype based on HLA is desired for a specific developmental project). Engrafting from a small number of HSC by a blood draw enables this to be a resource for virtually all cancer patients. Secondly, since a large quantity of conditionally immortalized HSCs are generated in one batch, larger mice cohorts can be created at one time. Establishing large cohorts enables, for example, the screening for a given patient of multiple drugs or combinations with more controls and with more tumors per arm enhancing accuracy of the data (with conventional models we tend to use 5 mice per drug/combination, and some screening plans include 10 or more drugs or iterations). Large cohorts may also allow, for example, the testing of multiple doses or combinations of a given compound as part of a pharmaceutical screening project; this provides a time advantage for completion of drug screenings which can be critical either to meet a patient timeline (treating him/her with optimal drug within a reasonable timeframe) or a drug developmental timeline (saving drug patent time since enables to triage drugs faster). Thirdly, the stability of the HSCs enables safe preservation for subsequent studies in the future, this being a key factor since it allows experimental stability, reproducibility, and comparability. Fourthly, the fact that from one animal many others can be established, enables a multiplier effect of proven HSCs of a given phenotype (either being from an individual patient or corresponding to a HLA-defined type that can be critical for an immune therapy development).

In some aspects, the invention provides a xenochimaeric animal comprising conditionally immortalized heterologous hematopoietic stem cells (HSCs) from a heterologous animal and a solid tumor from the animal. In some aspects, the invention provides a xenochimaeric mouse comprising conditionally immortalized heterologous hematopoietic stem cells (HSCs) from a heterologous animal and a solid tumor from the animal. In some embodiments, the HSCs and the tumor are from the same individual; for example, a cancer patient. In some emodiments, the HSCs and the tumor are from different individuals; for example, HSCs from an individual without cancer and a tumor from a cancer patient. In some embodiments, the heterologous animal is human. In some embodiments, the heterologous animal is not human; e.g. a dog, a cat, etc.

### III. USES OF XENOCHIMAERIC ANIMALS (E.G. MICE)

### A. Drug Discovery

The invention provides methods for evaluating a test agent for treating cancer. In some embodiments, a test agent is administered to xenochimaeric animal in a population of xenochimaeric animals of the invention and the response of the tumor to the test agent is evaluated. In some embodiments, the xenochimaeric animal is a xenochimaeric mouse. In some embodiments, a test agent is administered to xenochimaeric mice in a population of xenochimaeric mice of the invention and the response of the tumor to the test agent is evaluated wherein the xenochimaeric mice are generated with human HSCs and human tumors or portions thereof.

In some embodiments, the test agent is administered to the xenochimaeric animals (e.g. mice) about two to about four weeks following introduction of the tumor to the animal. In some embodiments, the test agent is administered to the xenochimaeric animals after about any one of one, two, three, four, five, six, seven, eight, nine or ten weeks following introduction of the tumor t the animal.

In some embodiments, the evaluation of the response of the tumor to the test agent is an evaluation of the size of the tumor which is evaluated by caliper or radiologic volume assessment over time with a given frequency (2-3 measurements per week if caliper, 2-3 measurements per month if with ultrasound or scan) wherein a decrease in the size of the tumor indicates therapeutic efficacy. In some embodiments, a decrease is the size of the tumor that indicates therapeutic efficacy is a decrease in size by more than about 5%, 10%, 15%, 20%, 25%, 30%, 35%, 40%, 45%, 50%, 55%, 60%, 65%, 70%, 75%, 80%, 85%, 90%, 95% or 100%.

In some embodiments, the evaluation of the response of the tumor to the test agent is an evaluation of the growth rate of the tumor which is evaluated as percentage growth as a function of time wherein a decrease in the growth rate of the tumor compared to an untreated tumor indicates therapeutic efficacy. In some embodiments, a decrease in the growth rate of a tumor that indicates therapeutic efficacy is a decrease in the growth rate by more than about 5%, 10%, 15%, 20%, 25%, 30%, 35%, 40%, 45%, 50%, 55%, 60%, 65%, 70%, 75%, 80%, 85%, 90%, 95% or 100% compared to a tumor in a xenochimaeric mouse that did not receive that test agent.

In some embodiments, the evaluation of the response of the tumor to the test agent is an evaluation of the vascularization of the tumor measured for instance as number/density of vessels (stained with CD31 for example) in histologic sections of treated and control tumors wherein a change in the vascularization of the tumor, or changes in the maturity or functionality of the tumor vessels compared to an untreated tumor indicates therapeutic efficacy. In some embodiments, a change in the vascularization or functionality or maturity of a tumor that indicates therapeutic efficacy is a change in the vascularization or functionality or maturity by more than about 5%, 10%, 15%, 20%, 25%, 30%, 35%, 40%, 45%, 50%, 55%, 60%, 65%, 70%, 75%, 80%, 85%, 90%, 95% or 100% compared to a tumor in a xenochimaeric mouse that did not receive that test agent. In some embodiments, all or a portion of the tumor vasculature is human vasculature.

In some embodiments, the evaluation of the response of the tumor to the test agent is an evaluation of stromal cells from the heterologous animal in the tumor wherein a change in the number of stromal cells from the heterologous animal in the tumor compared to an untreated tumor indicates therapeutic efficacy. Stroma cells include, but are not limited to, macrophage, fibroblasts, myofibroblasts, endothelial cells, blood vessels, and/or lymph vessels. Change is stroma may be related to the entirety of the above series, or to a specific one. Change may mean increase or decrease compared to untreated controls. In some embodiments, a decrease in the number of stromal cells in the tumor that indicates therapeutic efficacy is a change in the number of stromal cells in the tumor by more than about 5%, 10%, 15%, 20%, 25%, 30%, 35%, 40%, 45%, 50%, 55%, 60%, 65%, 70%, 75%, 80%, 85%, 90%, 95% or 100% compared to a tumor in a xenochimaeric mouse that did not receive that test agent. In some embodiments, all or a portion of the stromal cells are human stromal cells.

In some embodiments, the evaluation of the response of the tumor to the test agent is an evaluation of immune cells from the heterologous animal in the tumor wherein a change in the number of immune cells from the heterologous animal in the tumor compared to an untreated tumor indicates therapeutic efficacy. The development and frequency of immune cells will be determined by staining samples with monoclonal antibodies and analyzing the stained cell populations by either flow cytometry and/or histology. For T cells, we will stain with antibodies to human CD45, CD3, CD4, CD8 and TCR; for B cells, anti-human CD45, CD19, IgM; for myeloid cells, human CD45, Mac-1, Gr-1, CD16, CD56, MHC Class II; for NK cells, human CD45, CD16, CD56; for NKT cells, CD45, CD3, CD4, CD8, CD16, CD56. Immune cells include, but are not limited to, dendritic cells, T cells, B cell, macrophages, NK cells, NKT cells, neutrophils, or basophils. Change is stroma may be related to the entirety of the above series, or to a specific one. Change may mean increase or decrease compared to untreated controls. In some embodiments, a change in the number of immune cells in the tumor that indicates therapeutic efficacy is a change in the number of immune cells in the tumor by more than about 5%, 10%, 15%, 20%, 25%, 30%, 35%, 40%, 45%, 50%, 55%, 60%, 65%, 70%, 75%, 80%, 85%, 90%, 95% or 100% compared to a tumor in a xenochimaeric mouse that did not receive that test agent. In some embodiments, all or a portion of the tumor immune cells are human immune cells. In some embodiments, the immune cells are dendritic cells, T cells, B cell, macrophages, NK cells, NKT cells, neutrophils, or basophils.

In some embodiments, the evaluation of the response of the tumor to the test agent is an evaluation of immune cells from the heterologous animal in the tumor wherein a decrease in the number of immune cells from the heterologous animal in the tumor compared to an untreated tumor indicates therapeutic efficacy. In some embodiments, a decrease in the number of immune cells in the tumor that indicates therapeutic efficacy is a decrease in the number of immune cells in the tumor by more than about 5%, 10%, 15%, 20%, 25%, 30%, 35%, 40%, 45%, 50%, 55%, 60%, 65%, 70%, 75%, 80%, 85%, 90%, 95% or 100% compared to a tumor in a xenochimaeric mouse that did not receive that test agent. In some embodiments, all or a portion of the tumor immune cells are human immune cells.

In some embodiments, the evaluation of the response of the tumor to the test agent is an evaluation of the activation status of immune or stroma cells in the xenochimaeric mice. For example, the proportion of activated T or natural killer (NK) cells in the stroma may be determined as a function or proportion of the total number of the immune cells (i.e., as a ration of activated over total) after an intervention, including but not limited to radiotherapy, immunotherapy, or cytokine therapy. In other example activated T or NK cells may be exclusive events after said intervention, not being found in the untreated or control tumors. In some embodiments, a decrease would indicate efficacy and in some cases an increase would indicate efficacy. For instance an experiment where radiotherapy elicits the expression of NK attractants and induces an immune anti-tumor reaction represents an example of where an increase in immune cells indicates or is associated with activity. An experiment where a cytokine elicits the inhibition of macrophages that sustain cancer cells and induces an anti-tumor reaction by depleting said macrophages represents an example of where a decrease in immune cells indicates or is associated with activity.

In some embodiments the evaluation of the response of the tumor to the test agent is an evaluation of the increase or decrease of the number of immune or stroma cells (or their activation level) as a function of a biologic modification or therapy. For example, an increase in stroma cells may indicate efficacy after the administration or tumor expression of a pro-homing cytokine. Alternatively, a decrease in homing of immune cells may indicate efficacy after the administration of a migration or adhesion inhibitor that could include a cytokine, small molecule, monoclonal antibody or protein.

In some embodiments, the evaluation of the response of the tumor to the test agent is an evaluation of the survival of the xenochimaeric mice bearing tumors, wherein an increase in the survival of the xenochimaeric mice bearing tumors compared to untreated xenochimaeric mice bearing tumors indicates therapeutic efficacy. In some embodiments, an increase in survival of xenochimaeric mice bearding tumors that indicates therapeutic efficacy is an increase in survival by more than about 5%, 10%, 15%, 20%, 25%, 30%, 35%, 40%, 45%, 50%, 55%, 60%, 65%, 70%, 75%, 80%, 85%, 90%, 95% or 100% compared a xenochimaeric mice bearing tumors that did not receive that test agent. In some embodiments the increase in survival is an increase in survival by days, weeks or months.

In some embodiments, the evaluation of the response of the tumor to the test agent is a combination of two or more of size of tumor, growth rate of tumor, vascularization of tumor, presence of stromal cells in tumor, presence of immune cells in tumor and survival of xenochimaeric mice bearing tumors.

In some embodiments, the test agent is an anti-cancer agent. Examples of anti-cancer agents include, but are limited to, e.g., chemotherapeutic agents, growth inhibitory agents, cytotoxic agents, agents used in radiation therapy, anti-angiogenesis agents, apoptotic agents, anti-tubulin agents, and other agents to treat cancer, anti-CD20 antibodies, platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets PDGFR-beta, BlyS, APRIL, BCMA receptor(s), TRAIL/Apo2, and other bioactive and organic chemical agents, etc. Combinations thereof are also included in the invention.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” refers to a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Nicolaou et al., Angew. Chem. Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANETM), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®)), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylomithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g., celecoxib or etoricoxib), proteosome inhibitor (e.g., PS341); bortezomib (VELCADE®); CCl-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releaseing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

A “growth inhibitory agent” when used herein refers to a compound or composition which inhibits growth of a cell. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

In some embodiments, the test agent is administered, for example but not limited to the following routes: parenteral administration, oral administration, intraperitoneal administration, intravenous administration, intramuscular administration, interstitial administration, intradural administration, epidural administration, intraarterial administration, subcutaneous administration, intraocular administration, intrasynovial administration, transepithelial administration, transdermal administration, pulmonary administration via inhalation, opthalmic administration, sublingual administration, buccal administration, topical administration, ophthalmic administration, dermal, ocular administration, rectal administration, vaginal administration and nasal inhalation via insufflation or nebulization. Various dosing schedules including but not limited to single or multiple administrations over various time-points, bolus administration, and pulse infusion are contemplated herein.

### B. Tumor Biology

The invention provides methods for evaluating the stroma of tumors. In some embodiments, the method comprises removing a tumor from a xenochimaeric mouse prepared by the methods described above and detecting the presence of stroma from the heterologous animal in the tumor. In some embodiments, the method comprises removing a human tumor from a xenochimaeric mouse prepared with human HSCs by the methods described above and detecting the presence of human and mouse stroma in the tumor. In some embodiments, the stroma is one or more of T cells, B cell, macrophages, dendritic cells, NK cells, NKT cells, neutrophils, basophils, endothelial cells, epithelial cells or fibroblast cells. Methods of detecting stroma, e.g. human and/or mouse stroma, are known in the art and examples are provided herein. In some embodiments, the presence of stroma is by determined by histochemistry. In some embodiments, the presence of stroma; e.g. human and/or mouse stroma, is determined by fluorescence in situ hybridization (FISH). In some embodiments, the presence of stroma; e.g. human and/or mouse stroma, is determined by fluorescence activated cell sorting (FACS). In some embodiments, the presence of stroma, e.g. human and/or mouse stroma, is determined by detecting nucleic acid specific for the stroma from the heterologous animal and/or mice. In some embodiments, the presence of stroma; e.g. human and/or mouse stroma, is determined by detection of the activity of the stroma from the heterologous animal and/or from the mouse. In some embodiments, the presence of human stroma is determined by detection of the activity of the human stroma; for example, but not limited to secretion of human stroma factors. In some embodiments, the human stroma factors are SDF1 (e.g. SDF-1a/CXCL12a and/or SDF-1/CXCL12b) or HGF. In some embodiments, the presence of CD151+, CD31+, LY1+, CD 45+, CD3+, CD19+, CD68+, CD4+, SMA+, and/or CD57+ from the heterologous animal; e.g. human, indicates the presence of stroma from the heterologous animal; e.g. human. In some embodiments, the presence of CD45 from the heterologous animal; e.g. human, indicates the presence of stroma from the heterologous animal; e.g. human.

In some embodiments, the invention provides methods to analyze the activity of tumor stroma in response to administration of an agent to xenochimaeric mice bearing tumors. In some embodiments, the xenochimaeric mice are generated using human HSCs and human tumors. In some embodiments, the agent is an anti-cancer agent or a candidate anti-cancer agent. In some embodiments, the anticancer agent or candidate anticancer agent is a small molecule, a polypeptide, a nucleic acid, an antibody, a monoclonal antibodies conjugated to one or more toxins, a decoy receptor, a gene-mediated therapy, a natural immune modulator, a synthetic immune modulator, a vaccine or radiotherapy.

### C. Use of Xenochimaeric Mice in a Method of Determination of Treatment

The invention provides methods of treating a cancer patient by administering an effective amount of an anticancer agent to the cancer patient, where the anticancer agent was shown to be effective in delaying or inhibiting the growth of a tumor in one or more xenochimaeric animals prepared with a tumor sample from the patient. In some embodiments, the xenochimaeric animals are xenochimaeric mice. In some embodiments, the xenochimaeric mice are prepared by the methods described above. In some embodiments, the xenochimaeric animals (e.g. mice) are pre-established xenochimaeric mice. In some embodiments, the xenochimaeric animals (e.g. mice) are generated with HSCs (e.g. conditional HSCs) from the patient. In some embodiments, the patient is a human. In some embodiments, the patient is a nonhuman animal such as a pet, a farm animal, a companion animal, and the like. In some embodiments, the anticancer agent was shown to be effective in delaying or inhibiting the growth a tumor in a population of xenochimaeric mice prepared with HSCs from the patient and a tumor sample from the patient according to the methods described above.

In some embodiments, the invention provides methods of treating cancer in a patient comprising administering an effective amount of an effective anticancer agent to the cancer patient, wherein the anticancer agent had been shown to be effective in delaying or inhibiting the growth of the tumor in a xenochimaeric animal (e.g. mouse) according to the method comprising

a) introducing HSCs from the patient to the mouse,

b) introducing a portion of malignant tumor from the patient to the mouse,

c) administering a candidate effective anticancer agent to the mouse,

d) analyzing the xenochimaeric mice for effective anticancer activity; where an effective anticancer agent is one that delays or inhibits the growth of the tumor in the xenochimaeric mouse compared to a the growth of a tumor in a xenochimaeric mouse that was not treated with a candidate anticancer agent. In some embodiments, the tumor sample is introduced to the mouse by engrafting the tumor subcutaneously, orthotopically or by a hematogenous route. In some embodiments, the method comprises introducing blood cancer cells to the xenochimaeric mice in place of a tumor or portions thereof. In some embodiments, the cancer is a head and neck cancer, a melanoma, a brain cancer, a respiratory tract cancer, an endocrine cancer, a breast cancer, a prostate cancer, a colorectal cancer, a gastrointestinal cancer, an osteosarcoma, a myeloblastoma, acute lymphoblastic leukemia (ALL), acute myelogenous leukemia (AML), chronic lymphocytic leukemia (CLL), chronic myelogenous leukemia (CML), or non-Hodgkin's lymphoma (NHL).

In some embodiments, the xenochimaeric animals (e.g. mice) are generated using conditionally immortalized HSCs. In some embodiments, the cells are subject to a protooncogene product that promotes cell survival and/or proliferation and to a polypeptide that inhibits apoptosis of the cell. In some embodiments, the protooncogene product is a Myc family polypeptide. In some embodiments, the polypeptide that inhibits apoptosis is a Bcl-2 family polypeptide.

In some embodiments, a xenochimaeric animals (e.g. mice) comprising heterologous HSCs and heterologous tumors wherein the heterologous HSCs and heterologous tumor is from the same individual (e.g. a human cancer patient or a non-human cancer patient). In some embodiments, a population of xenochimaeric animals (e.g. mice) comprising heterologous HSCs and heterologous tumors wherein the heterologous HSCs and heterologous tumor is from the different individuals. For example, the heterologous HSCs may be from a human that does not have cancer and the heterologous tumor is from a human cancer patient. In some embodiments, the heterologous HSCs and heterologous tumor from different individuals is matched in one or more characteristics. For example, the heterologous HSCs and tumor may be matched for one or more cell surface markers; e.g. an HLA marker as described above.

In some embodiments, the HSCs are introduced to the mice about 8 to about 10 weeks prior to introduction of the tumor to the mice. In some embodiments, blood samples from the mice are analyzed for the presence of human cells prior to introduction of the tumor to the mice.

In some embodiments, the candidate anticancer agent is administered to the xenochimaeric mice about two to about four weeks following introduction of the tumor to the mice.

In some embodiments, the anticancer agent is a small molecule, a polypeptide, a nucleic acid, an antibody, a monoclonal antibodies conjugated to one or more toxins, a decoy receptor, a gene-mediated therapy, a natural immune modulator, a synthetic immune modulator, a vaccine or a radiotherapy. In some embodiments, the anticancer agent is used in combination with a chemotherapy, a radiotherapy and/or an immune therapy.

In some embodiments, the anticancer agent is administered, for example but not limited to the following routes: parenteral administration, oral administration, intraperitoneal administration, intravenous administration, intramuscular administration, interstitial administration, intradural administration, epidural administration, intraarterial administration, subcutaneous administration, intraocular administration, intrasynovial administration, transepithelial administration, transdermal administration, pulmonary administration via inhalation, opthalmic administration, sublingual administration, buccal administration, topical administration, ophthalmic administration, dermal, ocular administration, rectal administration, vaginal administration and nasal inhalation via insufflation or nebulization. Various dosing schedules including but not limited to single or multiple administrations over various time-points, bolus administration, and pulse infusion are contemplated herein.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further details of the invention are illustrated by the following non-limiting Examples. The disclosures of all references in the specification are expressly incorporated herein by reference.

### EXAMPLES

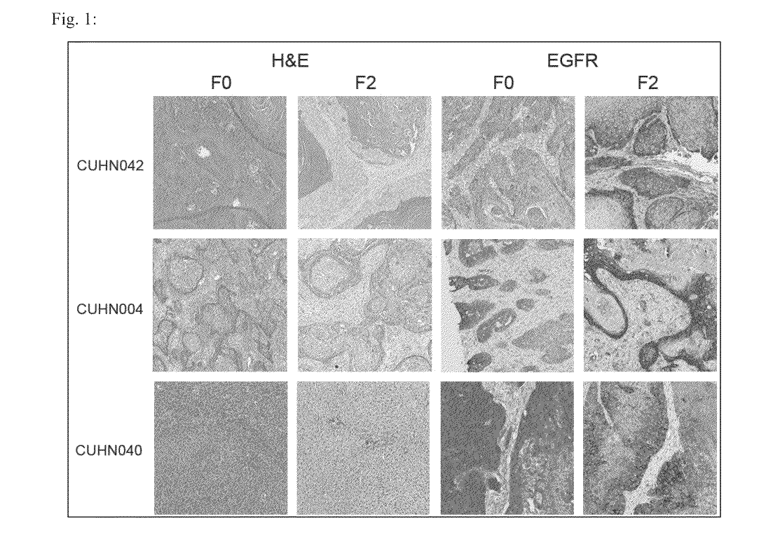

The examples below are intended to be purely exemplary of the invention and should therefore not be considered to limit the invention in any way. The following examples and detailed description are offered by way of illustration and not by way of limitation. FIG. 1 shows examples of tumors from standard implantation models and original patient tumors as reference.

The limitations of cancer cell lines in biological characterization and drug screening have led to the development of direct patient tumor xenograft models. However, tumors implanted in immunocompromised mice show genetic drift in microenvironment genes, and the unnatural interplay between mouse stromal cells and human cancer cells limits their value. To overcome this, the inventors have developed a technique to immortalize, stabilize, and expand human hematopoietic stem cells (HSCs), and reconstitute the hematopoietic system of a mouse on which a patient's tumor is then transplanted to create xenochimaeric mice. The human HSCs give rise to stromal and immune cells that home into the tumor and reconstitute its microenvironment. Even after extensive passage on nude mice, the transcriptome of these xenochimaeric mice tumors aligns more closely to that of the patient tumor than those grown on nude or NSG non-humanized control mice. Similarity is most pronounced in expression of epithelial, stromal, and immune genes. Differential tumor response to radiation treatment (RT) indicates that these histological and genomic features produce tangible changes in tumor development. The data show that these xenochimaeric mice can help recapitulate the tumor microenvironment, help reverse the initial genetic drift after regular mouse passaging, and provide a more accurate model to direct patient treatment and drug screening.

### Example 1

### Improved Production of Purified Recombinant Tat-MYC and Tat-Bcl-2 Fusion Proteins

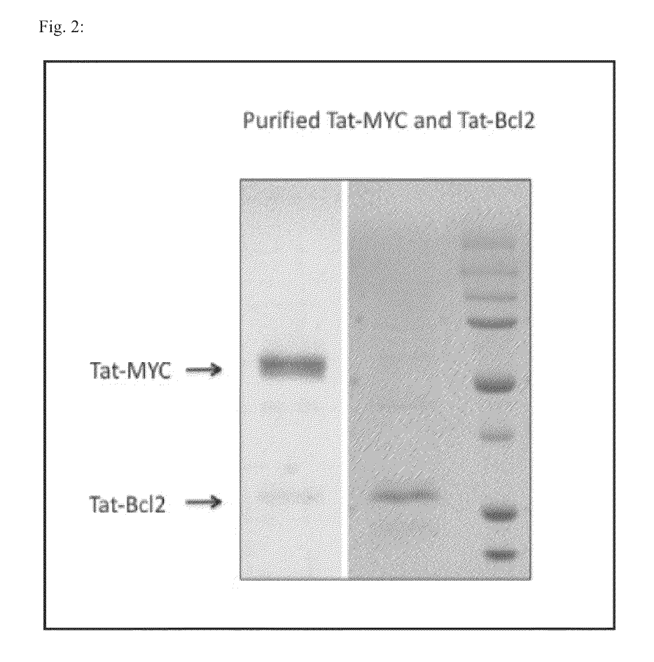

DNA encoding for Tat-MYC and Tat-Bcl-2 fusion proteins (human MYC and human Bcl-2 each fused to Tat from HIV-1) were cloned into bacterial expression plasmids (Invitrogen Gateway pDEST15 vector). The expression plasmids were transformed into Escherichia coli and overexpression was induced with IPTG. Induced cells were lysed in the presence of urea and the lysate was clarified and run on a nickel-affinity column. Fractions containing protein were dialyzed to remove the urea before being run on a size-exclusion column. Relevant fractions were pooled and endotoxins were further removed using an ActiClean column. A sample of the protein was run on a 15% SDS-PAGE gel and stained with a silver stain (FIG. 2).

### Example 2

### Biological Assay for Purified Tat-MYC and Tat-Bcl-2 Fusion Proteins

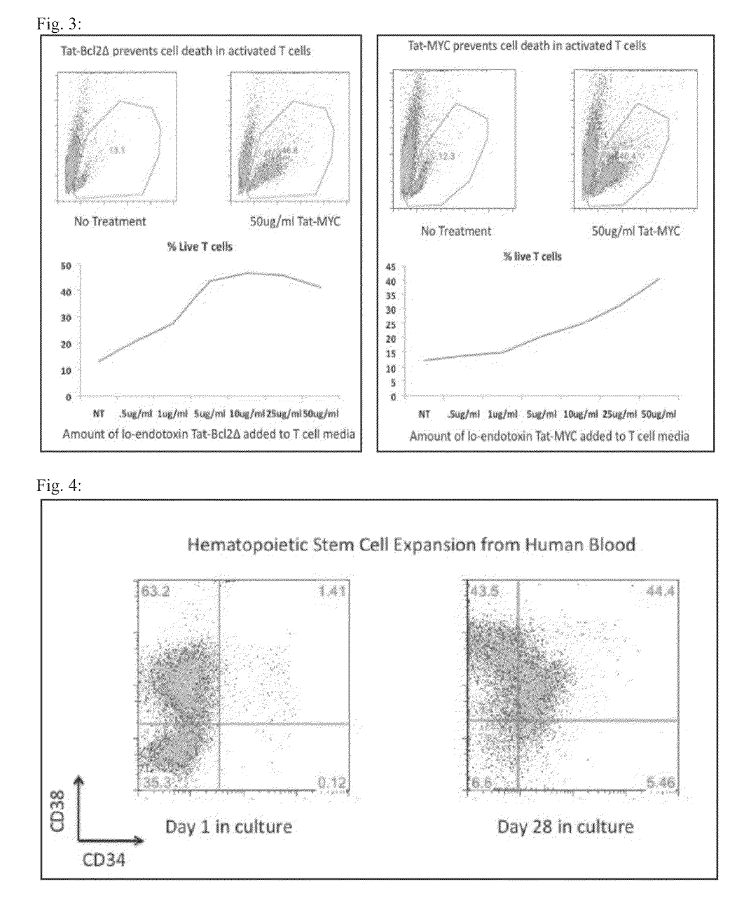

Murine CD4⁺ T-cells were isolated from peripheral blood of mice injected with 10 ng/ml G-CSF. The stem cell population was activated with antibodies to CD3 and CD28 for 72 hours in stem cell media (Stemline II), typically 1 μg/ml each, supplemented with recombinant IL-3, IL-6 and stem cell factor (SCF). Cells were washed to remove the cytokines and live cells enriched on a Ficoll-Hypaque gradient. Live cells were then incubated in either media alone or media supplemented with either Tat-Myc (50 μg/mL) and varying amounts of Tat-Bcl-2 (0-50 μg/mL), or Tat-Bcl-2 (50 μg/mL) and varying amounts of Tat-MYC (0-50 μg/mL). For each concentration of protein, the percentage of live T-cells was determined by FACS through gating on the live cell population by forward and side scatter, as well as staining for live cells by exclusion of 7-aminoactinomycin D (7AAD) (FIG. 3). A similar experiment was conducted using B-cells in place of CD4⁺ T-cells. B-cells were stimulated with IgM and CD40 in place of CD3 and CD28, with other parameters and results of the experiment generally the same.

### Example 3

### In Vitro Generation of Ctlt-HSCs Using Tat-MYC and Tat-Bcl-2

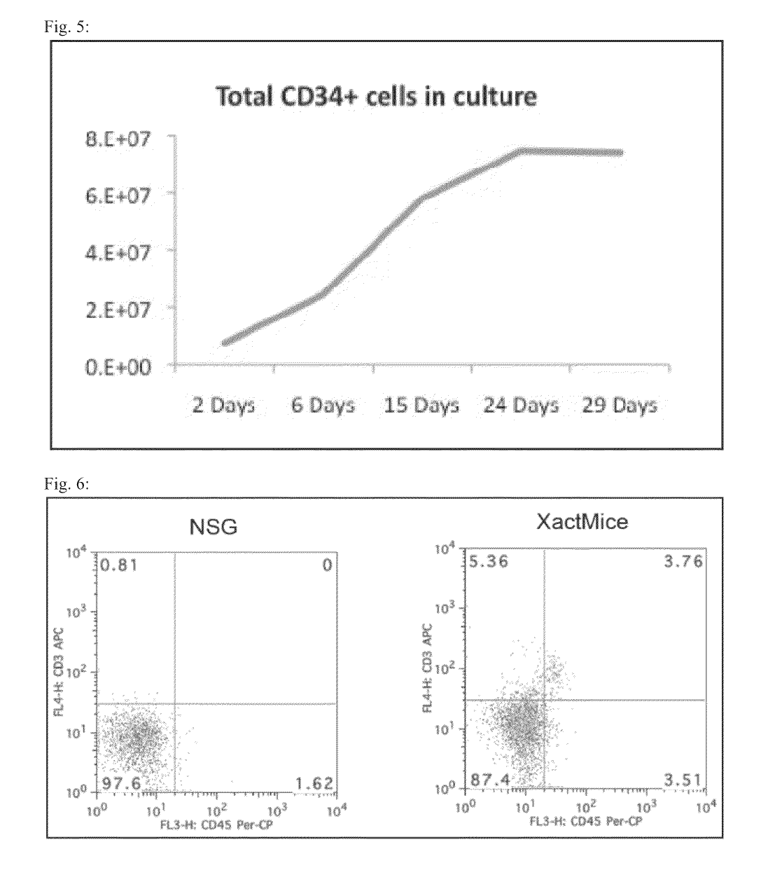

Human cord blood cells were used as a source to produced ctlt-HSCs. Red blood cells were lysed by incubating the cord blood cells in a hypotonic lysis buffer. The remaining cells were cultured at 2×10⁶ cells/ml in Stem line media (Stem Cell Technologies) supplemented with IL-3, IL-6 and SCF as well as 20 mg/mL of each Tat-MYC and Tat-Bcl-2. Cells were incubated in 24 well plates in 1 mL of medium, with a starting density of 2×10⁶ cells per well. The medium was replaced every two days. After 14 days, FACS data was collected to observe an increase in percentage of CD38⁺, CD34⁺ HSCs from 1.5% to 44.4%, demonstrating the expansion of lt-HSCs after incubation with recombinant purified Tat-MYC and Tat-Bcl-2 (FIG. 4). The total CD34⁺ HSCs in the culture was also regularly monitored, with regular increase in the total number of lt-HSCs demonstrating the formation of ctlt-HSCs (FIG. 5). Importantly, the total number of human lt-HSCs is increased steadily in the cultures over the period in which they were analyzed. The HSCs obtained from this culture on day 28 were used to reconstitute the mice discussed in Example 4.

### Example 4

### Formation of Xenochimaeric Mice

Cohorts of xenochimaeric mice were generated using donor human cord blood progenitors. Human ctlt-HSCs generated as described in Example 3 were washed in phosphate buffered saline and resuspended in phosphate buffered saline (PBS). The ctlt-HSCs (typically ˜10⁶/mouse) were then intravenously injected into sublethally irradiated (300 rads) NSG mice seven days after irradiation. Peripheral blood samples were taken from the xenochimaeric mice every four weeks after transplantation and analyzed using FACS for human CD45⁺ and human CD3⁺ cells. FIG. 6 shows scatterplots of FACS analysis used to assess mouse blood for human CD45⁺ and CD3⁺ cells with (right panel) and without (left panel) implantation of ctlt-HSCs into a NSG mouse. Xenochimaeric mice were fully constructed about 10 to 12 weeks after the ctlt-HSCs were injected into the sublethally irradiated NSG mice.

### Example 5

### Implantation of a Human Tumor onto Xenochimaeric Mice

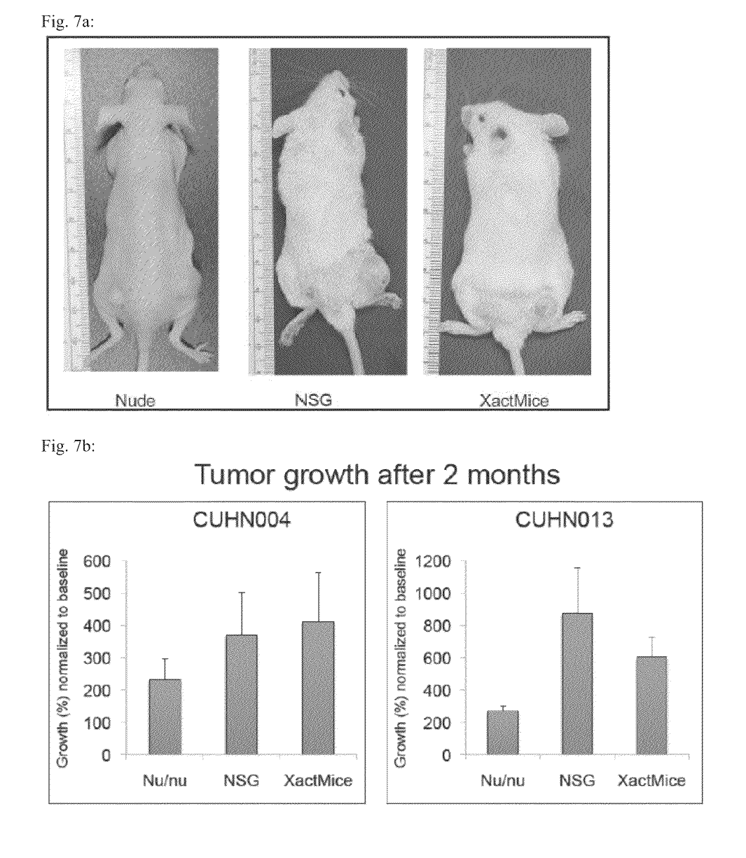

Two samples of an early passage (F2) tumors from the head and neck squamous cancers CUHN004 and CUHN013 were cut into small pieces measuring 20-25 mm³ and implanted subcutaneously on the flank of approximately five nu/nu, five NSG and five xenochimaeric mice (where the immune system was heterologous to the implanted tumor) (FIG. 7a).

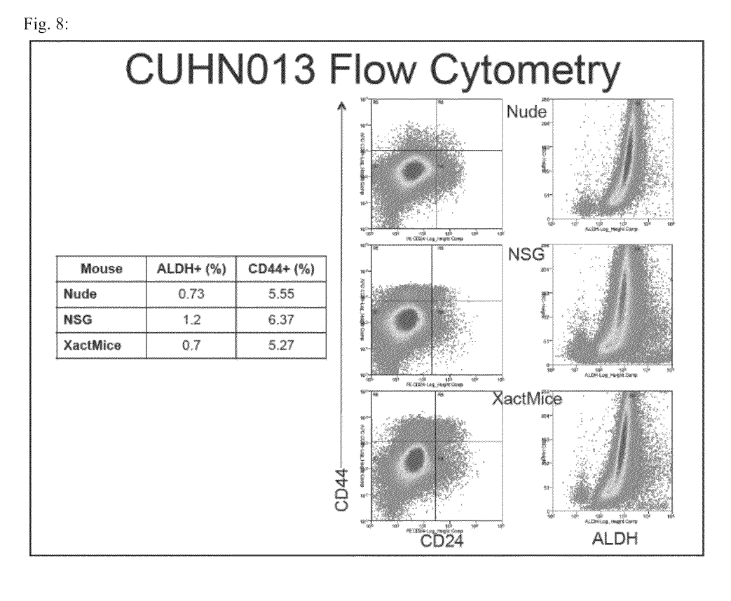

The growth of the tumor was monitored for two months after engraftment (FIG. 7b). No definitive trend was observed in the growth rates of the three types (nu/nu, NSG, and xenochimaeric mice). Cancer stem cells (CSCs) for each mouse was analyzed by FACS for CD44⁺,CD24⁺ cells, and ALDH⁺ cells for differences between the profiles of the nu/nu, NSG and xenochimaeric mice with engrafted tumors (FIG. 8). There was little significant difference of tumor size between the mouse strains.

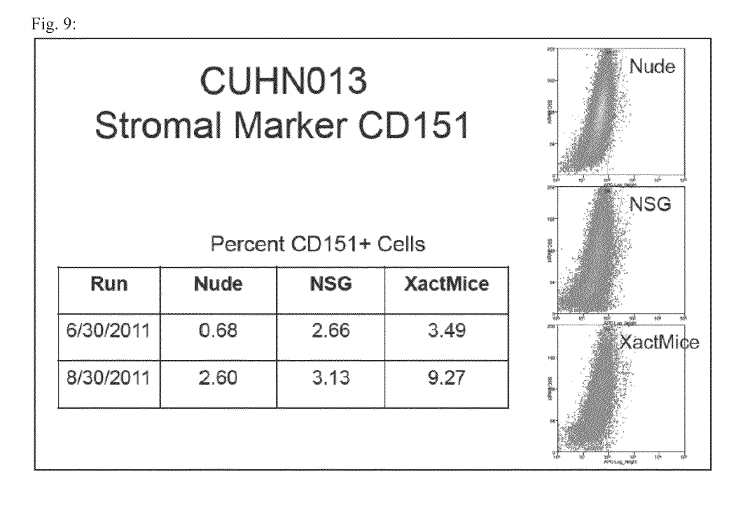

In order to demonstrate progenitor homing from the humanized bone marrow to the tumor, the stroma of the engrafted tumors were compared between the mouse strains by measuring the percentage human CD151⁺ cells one month and three months after tumor engraftment. The xenochimaeric mice averaged a three-fold increase in human CD151⁺ cells relative to the nu/nu and NSG mice three months after engraftment, indicating significantly more human stromal elements (FIG. 9). This increase is particularly striking because the tumors increased in volume from the 1-month to the 3-month period by at least a 3-fold, so the absolute increase in CD151⁺ cell count was 10-fold in the xenochimaeric mice overall.

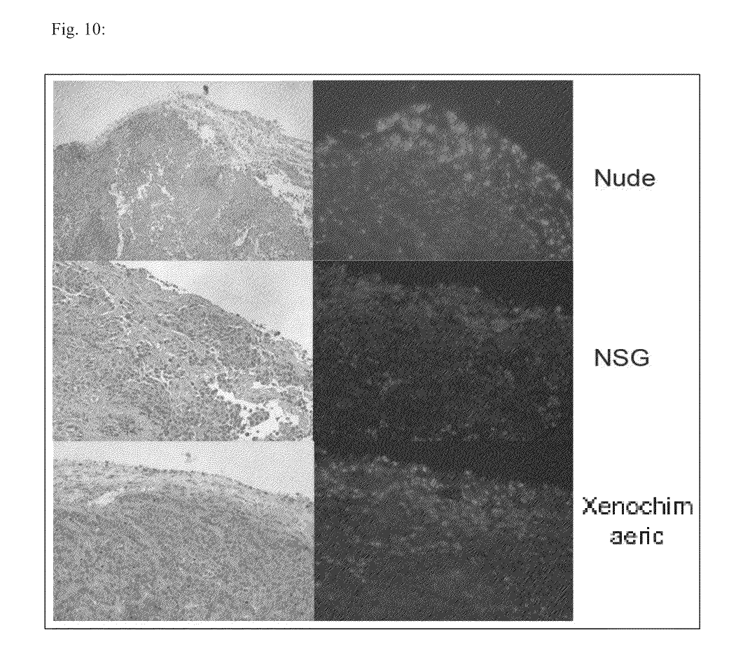

Human stromal elements were visualized using a differential fluorescence in situ hybridization (FISH) DNA staining assay of the engrafted tumors (FIG. 10). By staining human DNA as red and mouse DNA as green using human and mouse Cot-1 DNA immunofluorescence probes, a semi-quantitative assessment of the ratio of human-to-mouse cells verified more human stromal elements and less mouse stromal elements compared to the NSG mice.

The origin of CD151⁺ cells collected by cell sorting was determined by DNA fingerprinting short tandem repeat (STR) analysis using primer sets for the human thyroid peroxidase (TPDX) and von Willebrand Factor type A (vWA) loci. The STR fingerprint obtained in this analysis was compared to that obtained by amplification of previously isolated mouse genomic DNA and genomic DNA from the patient tumor originally grafted into the xenochimaeric mouse. PCR banding patterns observed from the CD151⁺ cells were different from the banding patterns of the patient tumor, indicating the stromal cells originated in the xenochimaeric mouse humanized bone marrow (FIG. 11).

### Example 6

### Autologous Tumor Engraftment on Xenochimaeric Mouse

In this experiment, CD34⁺ HSCs are harvested from the peripheral blood of patients with solid tumors (head and neck squamous carcinomas). Lt-HSCs are expanded using recombinant purified Tat-MYC and Tat-Bcl-2 as described in Example 3. The resulting ctlt-HSCs are used to reconstitute sublethally irradiated (300 rads) NSG mice, generating xenochimaeric mice similar to as described in Example 4. Early passage tumors (F2) from the same patient donating the CD34⁺ HSCs are implanted into the same xenochimaeric mouse.

The growth of the tumor is monitored for two months after engraftment. The stroma of the engrafted tumor is monitored by measuring the percentage human CD151⁺ cells one month and three months after tumor engraftment, and compared to NSG mice without a human reconstituted bone marrow (control mice).

Human stromal elements are visualized using a differential FISH DNA staining assay of the engrafted tumors. By staining human DNA as red and mouse DNA as green using human and mouse Cot-1 DNA immunofluorescence probes, a semi-quantitative assessment of the ratio of human-to-mouse cells can be used to verify more human stromal elements and less mouse stromal elements compared to the NSG control mice.

The origin of CD151⁺ cells from the autologous tumor engrafted xenochimaeric mouse collected by cell sorting is determined by DNA fingerprinting STR analysis using primer sets for TPDX and vWA loci. The STR fingerprint obtained in this analysis is compared to that obtained by amplification of previously isolated mouse genomic DNA and genomic DNA from the patient tumor originally grafted into the xenochimaeric mouse. Similar PCR banding patterns observed from the CD151⁺ cells compared to the banding patterns of the patient tumor, indicate that the stromal cells originate in the xenochimaeric mouse humanized bone marrow and from the same patient origin as the tumor itself.

Additional testing may involve GFP-tagging of the lt-HSCs to confirm the presence inside the tumor once implanted in the xenochimaeric mice, as evidence of GFP activity on stroma. Additional testing may involve testing for human chromosomes X and Y (XX for female donor and XY for male donor) in stroma spaces in the tumor to confirm human stroma homing. Additional tests may include crossing donors from HSCs and tumor (one female, one male) and conduct alternate XX and XY to detect both genotypes in stroma/tumor or tumor/stroma.

For the autologous tumor engraftment in the xenochimaeric mice, a decrease in the activity of T-cells, natural killer (NK) cells, and NKT-cells is seen relative to the heterologous tumor engraftment, resulting is decreased allogeneic reactivity and tumor rejection. Each one of these populations may be depleted with specific monoclonal antibodies followed by evaluating tumor growth, survival.

### Example 7

### Formation of Xenochimaeric Mice with HSCs from Patients Undergoing Chemotherapy

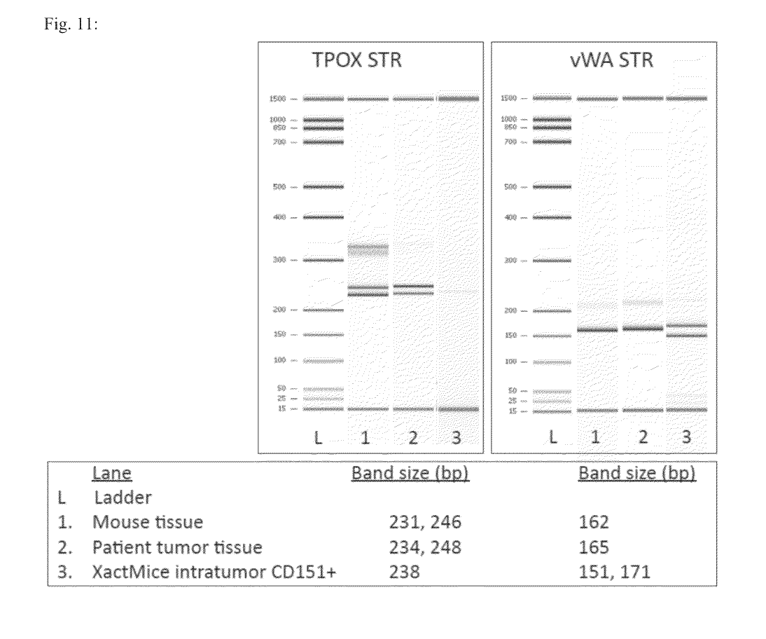

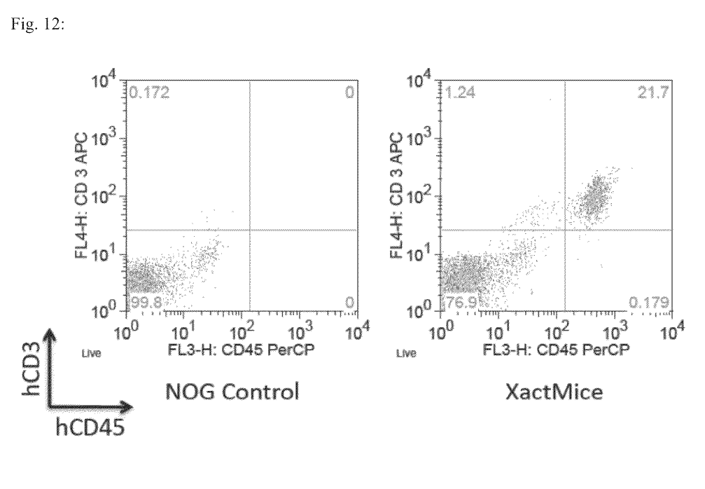

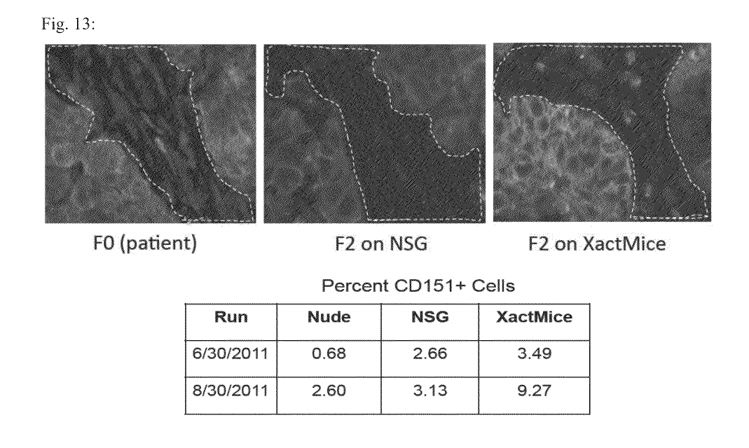

In this experiment, CD34⁺ HSCs were harvested from the peripheral blood of chemotherapy patients treated with G-CSF. Lt-HSCs were expanded using recombinant purified Tat-MYC and Tat-Bcl-2 as described in Example 3. The resulting ctlt-HSCs were used to reconstitute sublethally irradiated (300 rads) NSG mice with ˜10⁶ cells/mouse, generating xenochimaeric mice. Peripheral blood from these xenochimaeric mice was analyzed using FACS and contains mature human CD45⁺, CD3⁺ cells, demonstrating successful implantation (FIG. 12). Stromal elements of patient tumors (F0) were compared to tumors grafted onto NSG and xenochimaeric mice using immunohistochemistry staining for human CD151, a marker indicating human stromal elements (FIG. 13). The xenochimaeric mice with grafted patient tumor had reconstituted the human stromal elements.

### Example 8

### Homing of Multiple Human Stroma and Immune Lineages on Xenochimaeric Mice

Human HSCs are isolated, stabilized and engrafted in NSG mice bone marrow to generate Xenochimaeric mice as described in other Examples. When resected patient tumor is implanted on Xenochimaeric mice, HSC progeny migrate into the xenograft and differentiate into stromal cells. In this experiment, tumors from the patient, nude, NSG, and Xenochimaeric mice were compared, and human stromal cells within Xenochimaeric mice tumors were characterized.

The stroma that forms in a developing tumor has two primary origins: mesenchymal and immune cells originating from the bone marrow, and fibroblasts and other cell types from local tissues. The cell surface antigen CD151 has been previously characterized as a mesenchymal cell marker and is found on many different cell types. CD45 is a hematopoietic cell surface marker found on B and T cells, as well as on hematopoietic progenitors. Tumors were harvested periodically and their cellular makeup analyzed by flow cytometry.

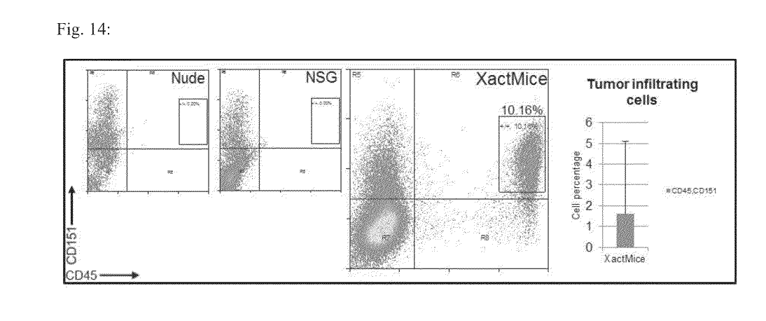

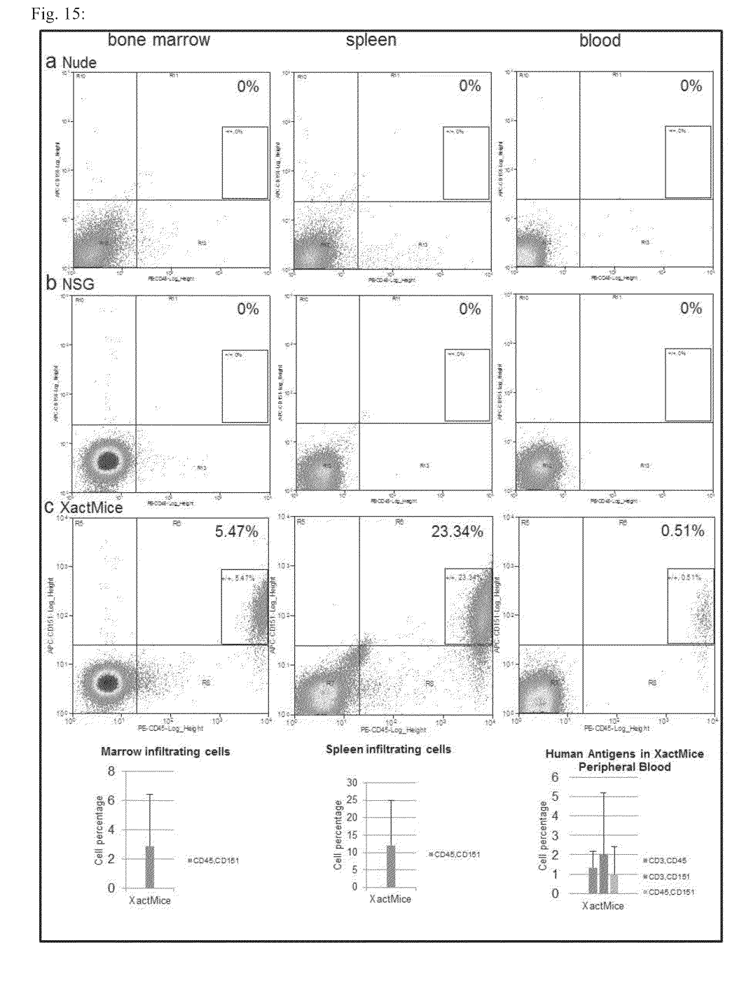

The data show that tumors grown in Xenochimaeric mice contain a unique population of cells displaying the human cell surface antigens CD45 and CD151, while tumors grown in the nude and NSG mice lack this population (FIG. 14). Likewise, cells removed from the bone marrow, spleen, and peripheral blood of the Xenochimaeric mice harbor similar double-positive populations, while cells from nude and NSG mice lack these markers (FIG. 15a-c).

### Example 9

### Human Stromal Cells Originate from Humanized Bone Marrow

Four different experiments using separate cohorts of Xenochimaeric mice were performed to confirm the origins of the human stromal cells. First, short tandem repeat (STR) analysis, a well-documented forensic examination, was used to compare highly variable DNA loci by PCR to establish the relationship between two or more DNA samples. DNA from CD45/CD151+ cells sorted from CUHN004 tumors grown on Xenochimaeric mice, and DNA from the originator CUHN004 F0 patient sample, were purified. The DNA was analyzed at two well-studied loci to identify STR polymorphisms (FIG. 16a). The data show a banding pattern from the CD45+/CD151+ cells that is distinct from the tumor DNA, indicating that the DNA comes from a unique and separate source. The following CD151 immunofluorescence analysis shown in FIG. 16 showed these were human, not mouse cells. Tumor xenografts from nude (NSG) and Xact Mice are composed primarily of human tumor cells surrounded by mouse stromal cells. Tumor cells were uniformly illuminated in both the NSG and Xenochimaeric mice tumors. Additionally, in the Xenochimaeric mice tumor, we detected individual human cells scattered throughout the otherwise unstained mouse stroma (FIG. 16b,c). While the mouse stroma within the nude xenograft (d) shows no evidence of human cell infiltration, human cells can be observed interspersed throughout the stroma within the Xenochimaeric mice xenograft (e; enlarged and highlighted with arrows.)

FISH analysis, using species-specific probes for highly repetitive Cot-1 DNA, was used to clearly identify the species of each cell. Tumor from a nude mouse was composed of human cells, encapsulated by and containing small islands of mouse stromal cells, as is typical of a xenograft tumor (FIG. 16d). In contrast, the tumor from the Xenochimaeric mice consisted of large bundles of human tumor cells, surrounded by bands of mouse stroma. Close examination of the stroma identified human cells interspersed throughout the mouse cells (FIG. 16e).

Xenochimaeric mice were generated using lt-HSCs from female cord blood. Following evidence of engraftment and humanization, tumors from a male patient were implanted. FISH analysis for X and Y chromosomes revealed tightly packed XY epithelial tumor cells surrounded by stromal cells in both nude and Xact Mice. In the NSG mouse (0, all tumor cells surrounding the stroma bind both probes, as befits their male origin. The stromal cells do not bind to either of the probes, indicating that they originate from the mouse (FIG. 160. In Xenochimaeric mice, the tumor cells are again male, but there is XX cell stroma invasion (FIG. 16g), in a pattern similar to that observed in the Cot-1 FISH analysis. The stroma is composed largely of mouse cells, but human cells containing two red-stained X chromosomes are interspersed among them. Detail inserts emphasize the XX cells both within the mouse stroma (whose nuclei tend to become granulated when stained with DAPI) and invading the adjacent tumor bundle. Taken together, these analyses provide proof that the donor HSC progeny have migrated from the hematopoietic system of Xenochimaeric mice after their progenitors' engraftment and are incorporated in the tumor.

### Example 10

### Stromal Cells within Tumors

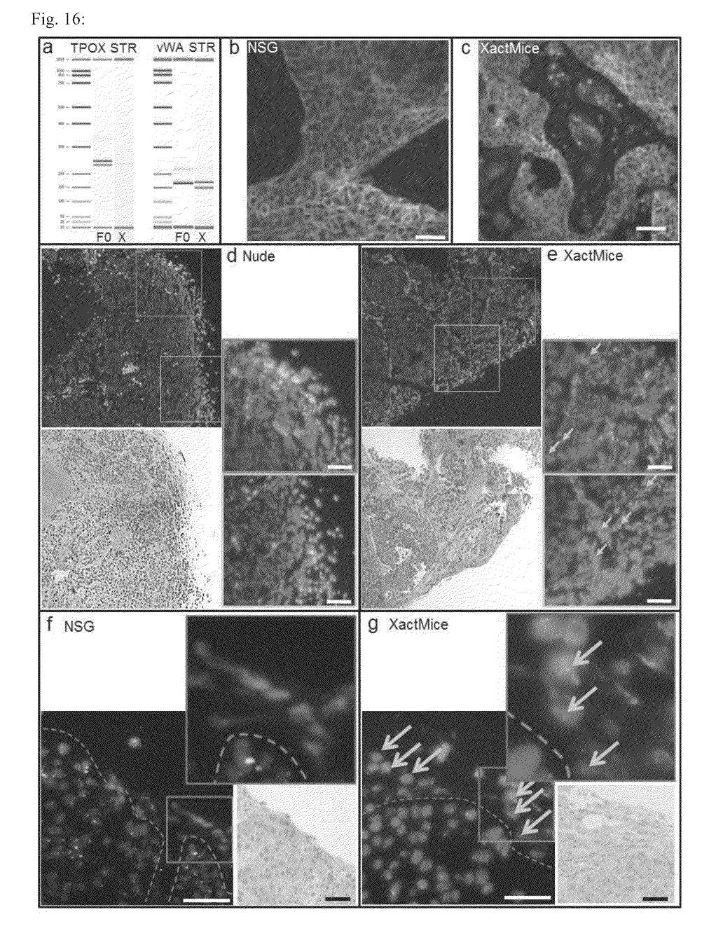

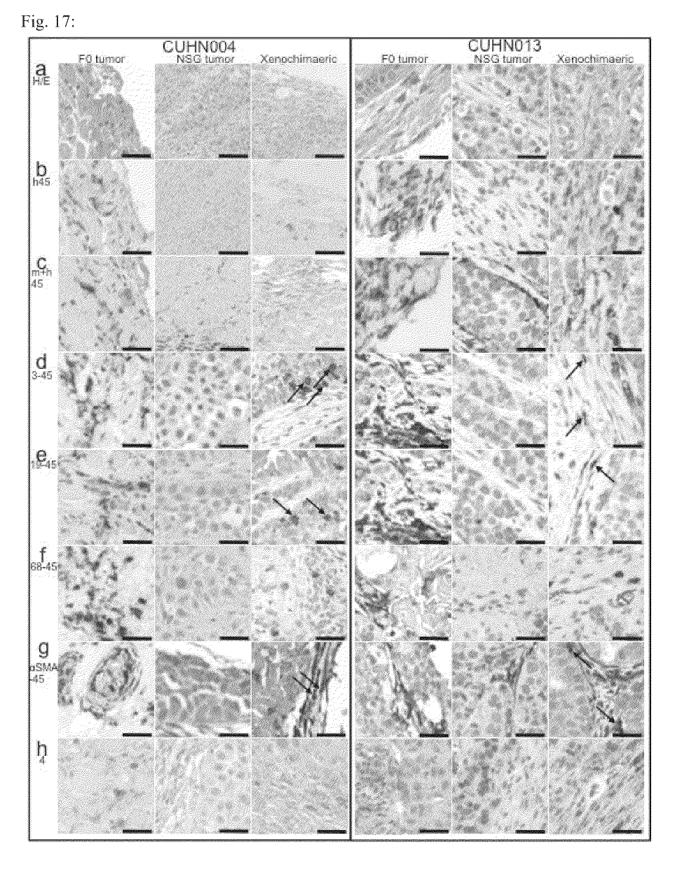

The types of human stromal cells within Xenochimaeric mice tumors, were studied using stained tumor sections. Sections from the CUHN004 and CUHN013 F0 patient tumors and corresponding NSG and Xenochimaeric mice xenografts generated as described in other Examples, were exposed to either human or human plus mouse pan-leukocyte CD45 antibodies (FIG. 17a-c). While the F0 tumors contained only human CD45+ cells (red) and the NSG tumors contained only mouse CD45+ cells (brown), the Xenochimaeric mice tumors contained both mouse and human CD45+ cells, indicating that HSC-generated white blood cells are invading these tumors. In the F0 tumors, staining for human CD45+ antibody is visible throughout the stroma and adjacent tumor tissue, and no mouse CD45+ staining is visible. In NSGs, no staining for human CD45+ antibody is visible, only mouse CD45+ cells, indicating that neither the human tumor cells nor the mouse stromal cells bind human CD45+ antibody. In Xenochimaeric mice, human CD45+ cells are found around the periphery of the tumor throughout the stroma, and within islands of tumor cells. Xenochimaeric mice, some cells within the tumor stroma and surrounding tissues are mouse CD45+, while a separate population of cells are human CD45+.

To more thoroughly characterize the lineages and fates of the infiltrating human cells, dual staining with human-specific antibodies to CD45 and either CD3 (T-cells), CD19 (B-cells), CD68 (macrophages), or alpha smooth muscle actin (aSMA; fibroblasts) was performed (FIG. 17. The human T-cell and B-cell populations identified by this process were similarly distributed throughout the F0 and Xenochimaeric mice tumors but absent in the NSG tumor. Macrophages were also present in F0 and Xenochimaeric mice tumors. However, in the F0 tumors they were sometimes marked by both the CD45 and CD68 antigens, while in the Xenochimaeric mice tumors, the CD68+ macrophages did not costain for CD45; in Xenochimaeric mice tumors, distinct CD68 and CD45 cell populations can be observed. The aSMA staining was not human-specific, as can be seen in the NSG tumors, but CD45/aSMA+ double-staining cells were identified in both the F0 and Xenochimaeric mice tumors, and they can be attributed to HSC differentiation. In F0 tumors, cells with either or both antigens (CD45/aSMA+) are present. In NSGs, some stromal cells stain for the aSMA antigen, indicating that this antibody crossreacts with mouse aSMA. However, since human CD45 is species-specific, Xenochimaeric mice tumor cells which stain for the presence of both antigens (indicated by red arrows) must be of human origin and exhibit some fibroblast characteristics.

Staining for human CD4+ cells showed their presence in Xenochimaeric mice tumors, indicating that the HSC-generated T-cells can continue to differentiate into T-helper cells (FIG. 17h). In Xenochimaeric mice, CD4+ cells are again observed in much the same pattern and locations as was seen in the F0 tumors.

Overall, homing of human CD151+, CD31+, LY1+, CD 45+, CD3+, CD19+, CD68+, CD4+, SMA+, and CD57+ cells indicate stroma (macrophage, fibroblast, endothelial cell) and immune (B-cell, T-cell, NK-cell) homing in Xenochimaeric mice tumors.

### Example 11

### Genetic Expression is Reconstituted in Tumors Grown on Xenochimaeric Mice

Since the Xenochimaeric mice model is designed to support xenograft tumor growth in a native environment, it is critical to demonstrate that the passaged tumors are genetically similar to the originator tumor. To this end, CUHN004 and CUHN013 tumor cells from tumors passaged in nude, NSG, and Xenochimaeric mice, as well as from the originator F0 flash-frozen patient tumors were micro-dissected. RNA was isolated and next generation sequencing was performed to compare gene expression between tumors.

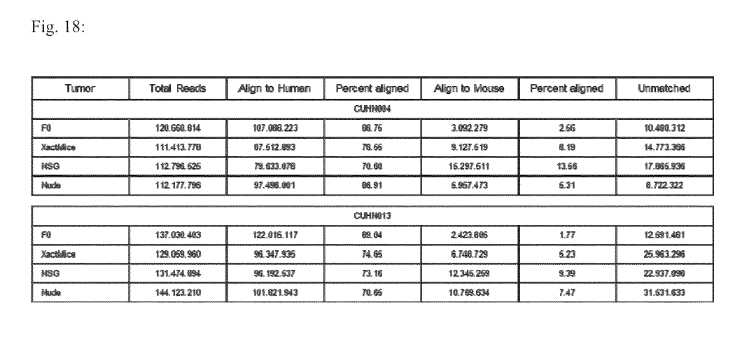

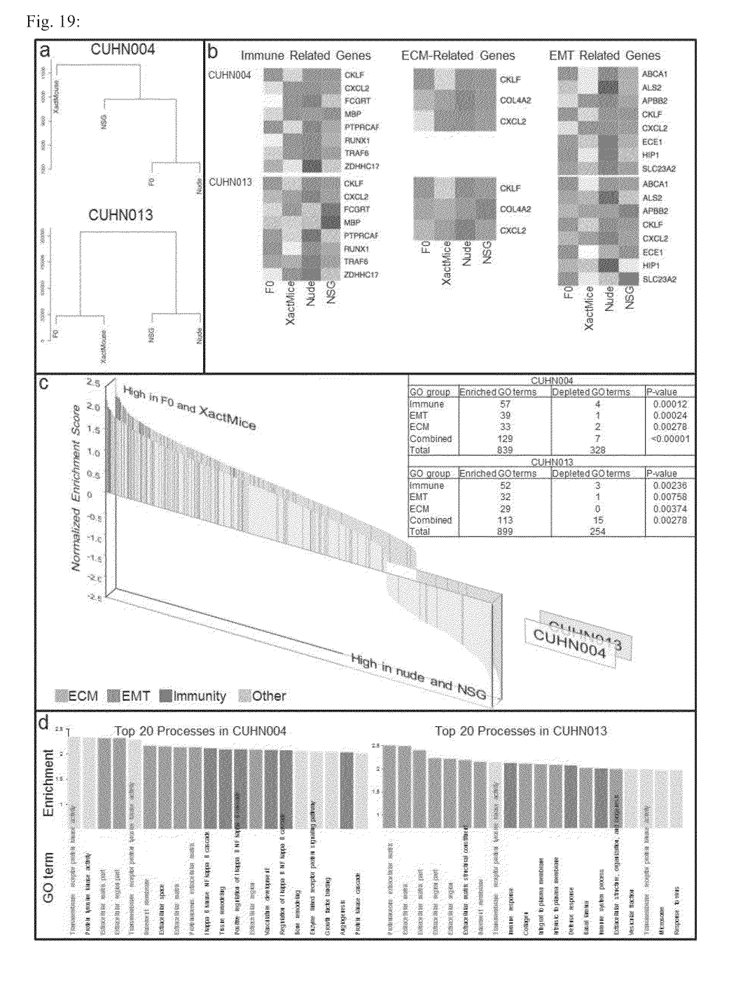

A summary of the sequencing data (FIG. 18) shows that the CUHN013 Xenochimaeric mice tumor transcriptome aligns more completely to the human genome than does the nude or NSG-originated tumors transcriptome. The tumor transcriptome from the CUHN004 Xenochimaeric mice aligns slightly less completely to the human genome than that from the corresponding nude mouse, but its alignment is more complete than that of the NSG mouse. Likewise, while the dendrogram of the CUHN013 shows that the Xenochimaeric mice and the F0 tumors are most similar, the dendrogram comparing RNA expression between the CUHN004 tumors indicates that the Xenochimaeric mice transcriptome clusters separately from all others, indicating a fundamental difference in its gene expression (FIG. 19). It is possible that these differences arise as a consequence of repeated tumor passage in nude mice, since the CUHN004 samples in question were implanted and analyzed as F14 tumors, while the CUHN013 samples were taken as F5 tumors. In this case, it may take multiple passages in the Xenochimaeric mice to completely revert its gene expression back to that observed in the F0 tumor.

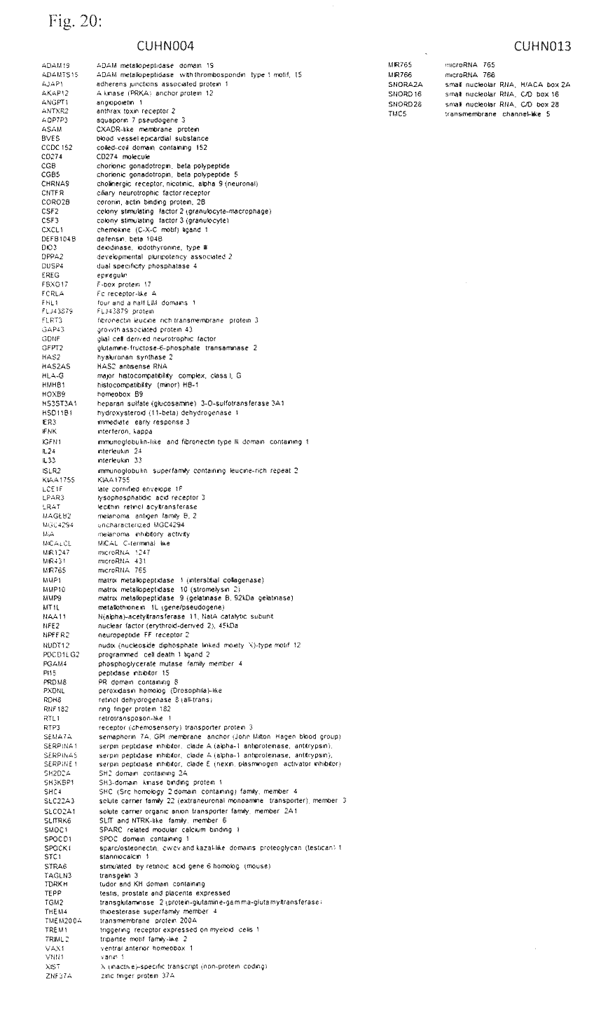

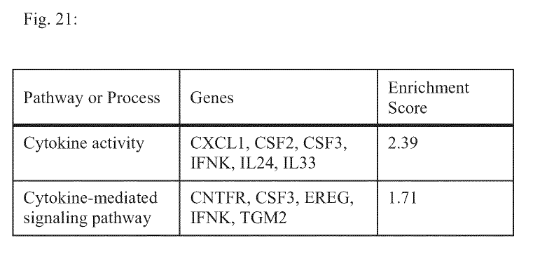

The transcriptome of the CUHN004 Xenochimaeric mice tumor contains 100 genes that are expressed at least five-fold more highly than in the F0, NSG, or nude tumors (FIG. 20). By comparison, the CUHN013 Xenochimaeric mice tumor transcriptome contains six such genes, five of which are snoRNAs or miRNAs. No F0, nude, or NSG CUHN004 or CUHN013 tumor transcriptome contains a comparably-sized group of overexpressed genes. Analysis of these genes indicates that they are heavily enriched for members of cytokine signaling pathways, particularly EGFR pathway components (FIG. 21). When this group of genes is excluded from the Xenochimaeric mice in dendrograms for each tumor, the relationship between the CUHN013 tumors does not change, but the CUHN004 expression is altered.

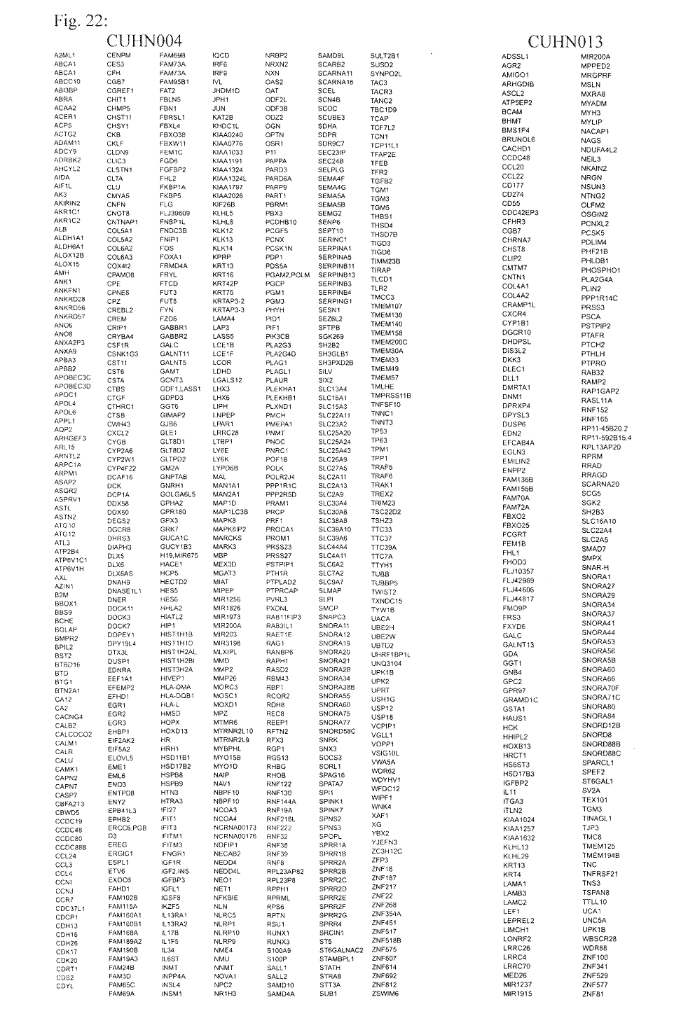

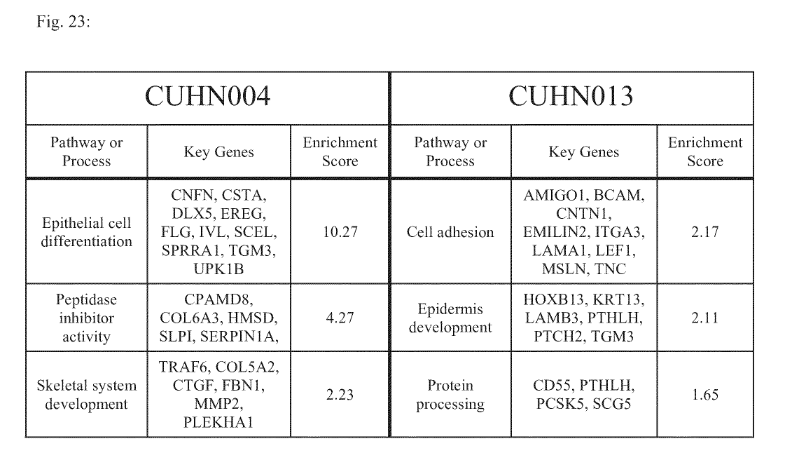

We next analyzed the CUHN004 and CUHN013 sequencing data to identify differentially expressed genes, whose expression is similar in the F0 and Xenochimaeric mice tumors but different from their expression in the nude and NSG tumors (FIG. 22). Although the variability between the CUHN004 and CUHN013 tumors resulted in few overlapping genes, many of the genes identified are common to several general biological processes, including epithelial differentiation, peptidase inhibition, cell adhesion, and protein processing (FIG. 23).

To identify specific features these processes alter in the Xenochimaeric mice, we captured all GO terms associated with the differentially expressed genes and used them to perform a gene set enrichment analysis (GSEA). Using this, we calculated an enrichment score for each of these GO terms. Many of the most enriched GO terms are associated with the immune system, the extracellular matrix (ECM), or in the epithelial-mesenchymal transition (EMT). We created heatmaps for both tumors, comparing the expression of several core genes linked to these GO terms (FIG. 19b), and we also generated a waterfall graph depicting the relative enrichment of all GO terms, highlighting those relevant to the above-indicated pathways (FIG. 19c) In CUHN004, thirteen of the top twenty most enriched GO terms are associated with the immune system, ECM, or EMT (p-value<0.00001); for CUHN013, fifteen of the top twenty fall within these categories (p-value<0.00001) (FIG. 19d). These are the types of processes that the Xenochimaeric mice are designed to replicate. Conversely, of the GO terms that are enriched in the nude and NSG tumors, few play a role in these processes.

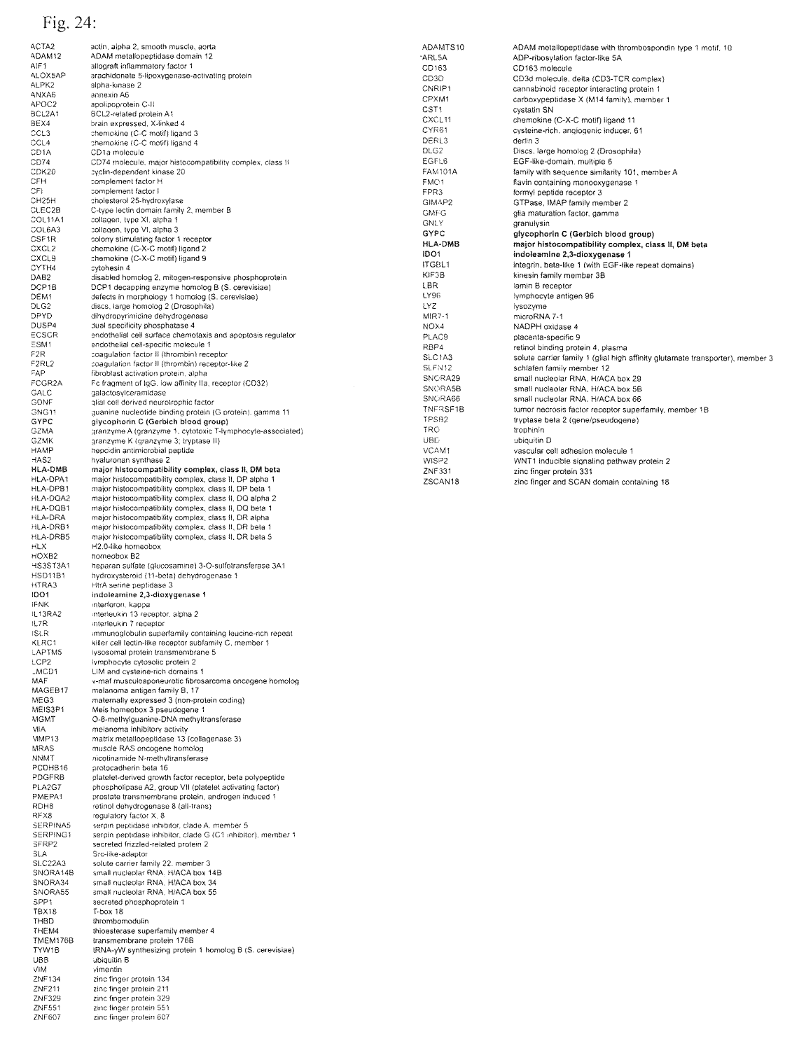

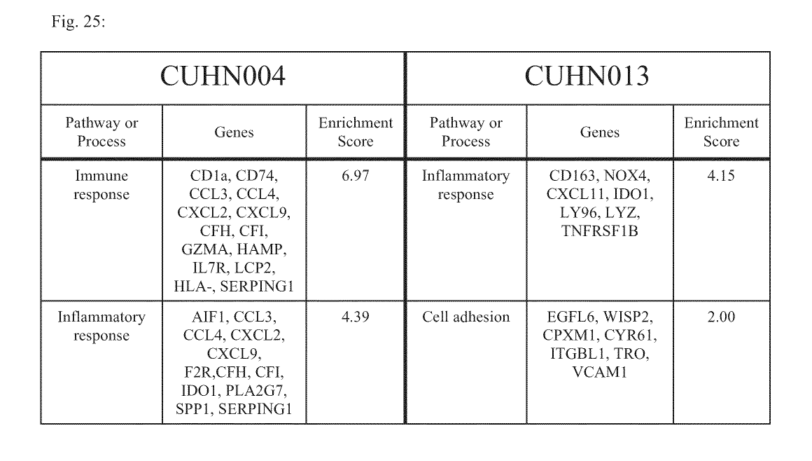

Finally, to clarify the relationship between differentially expressed genes and the enriched processes identified by their GO terms, we identified many differentially expressed genes expressed exclusively in the F0 and Xenochimaeric mice tumors (FIG. 24). Many of these genes play a role in the immune response or in EMT, or are components of the extracellular matrix, indicative of the lt-HSC derived invasive cells playing an active role in stromal growth (FIG. 25). In support of this, several such genes have been shown to be activated by stromal processes. Tumors grown in Xenochimaeric mice had a highly significant similarity to original F0 tumors especially in areas pertaining microenvironment and immune pathways, where they differed from tumors grown on nude or NSG mice.

### Example 12

### Xenochimaeric Mice Model for Treatment and Drug Screening

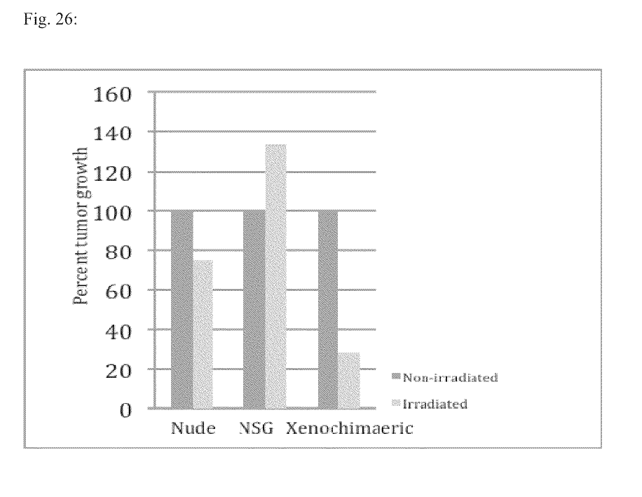

Cohorts of CUHN013 tumor-bearing nude, NSG, and Xenochimaeric mice were treated with radiation therapy (RT), which is standard of care for head and neck cancer management. The inflammation and subsequent cytokine release induced by RT is known to prompt an immune response. A total localized dose of 12 Gy ionizing radiation was administered to flank xenografts in four 3-Gy fractions over eleven days. Tumor growth and immune cell invasion was monitored during and after this period. The tumors in the nude and NSG mice did not respond to RT, while the tumors in the Xenochimaeric mice regressed markedly. (FIG. 26).

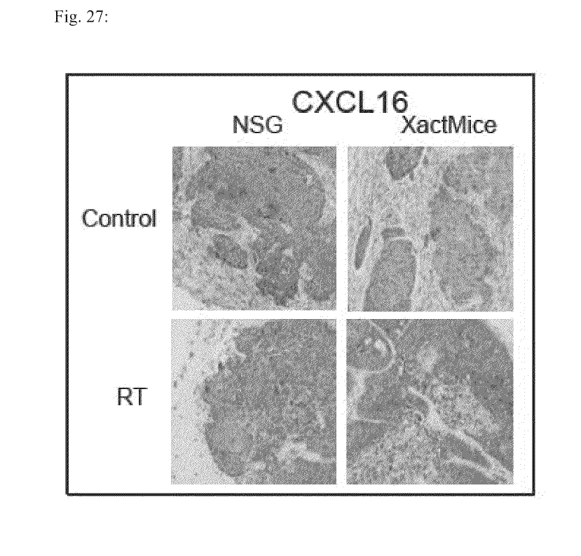

The CXCL16 chemokine is a known ligand for the CXCR6 receptor, found on activated T cells, natural killer (NK) cells, and cytotoxic T cells. It has been previously observed that tumor cells release CXCL16 when exposed to ionizing radiation. CXCL16 was shown to be expressed in CUHN013 by our RNA sequencing. The irradiated and non-irradiated NSG and Xenochimaeric mice tumors were examined by IHC for the presence of CXCL16. Although present in all tumors, it was significantly upregulated in the Xenochimaeric mice irradiated tumor (FIG. 27).

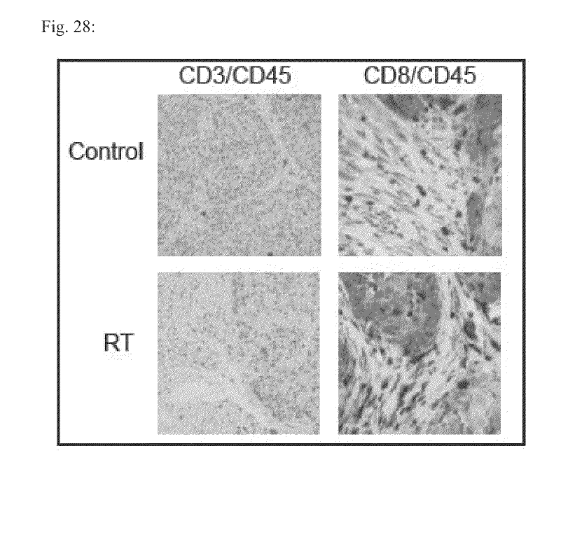

Flow cytometry and IHC for CD3/CD45-postive cells indicated that T cells invaded both the non-irradiated and irradiated Xenochimaeric mice tumors (FIG. 28). The tumors were also screened for the presence of the CD8 and CD45 antigens, expressed by cytotoxic T cells, and CD57 and CD45, expressed by NK cells, by IHC. In non-irradiated tumors, the majority of the CD45+ cells (in red), do not also stain for either CD8 or CD57 (in brown). However, in the irradiated tumors, most of the CD45+ cells are also positive for CD8 or CD57, indicating that these cytotoxic T cells are activated and being recruited to this site. Thus, it appears as if the human immune system in the Xenochimaeric mice can be activated by irradiation, enabling it to attack tumors outside of the field of radiation. Similar phenomena have been described in nonhumanized mouse models, as well as in human cancer patients, where they are known as abscopal effects.

In summary, genes whose products play a role in epithelial differentiation and whose products are often associated with the immune system, extracellular matrix, and epithelial mesenchymal transistion are expressed in Xenochimaeric mice at a level more similar to that observed in the F0 tumor. Further, tumor implantation in nude and NSG induces an inhibition in these microenvironment and immune-associated genes. Finally, we have shown that expression of these genes results in functional aspects of the immune system that respond to RT in a manner similar to that observed in cancer patients. We conclude that the Xenochimaeric mice system represents an important first step toward recapitulating a tumor environment more similar to that found in the patient. Further, the realization that Xenochimaeric mice tumor implantation reverses the initial genetic drift after regular mouse passaging highlights the strength of the approach, as it hints that given the right environment, tumors revert to their original state. This study arises from the central tenet that cancer cells will more faithfully retain their original phenotype and genotype when placed in an environment that mimics that in which they arose. The Xenochimaeric mice model represents a quantum leap toward this endeavor by creating an in vivo system in which tumor growth is at least partially regulated by a surrogate immune system derived from the same host. Although various types of humanized mice have been previously reported, none have attempted to recapitulate the patient's tumor growth within a native immune environment. Furthermore, because the lt-HSCs we use have been stabilized before infusion on recipient mice, the bone marrow from these mice can be purified for human HSCs and passed subsequently; thus, the model sustains itself, which is an additional improvement associated with our system. We stand at the dawn of the age of individualized medicine where new sequencing technology allows us to examine the genetic cause of many medical conditions. Cancer research has benefited significantly from this technology, which has increased our ability to customize therapy in response to mutated genetic pathways that drive an individual's tumor growth. However, tumors do not exist in the absence of the host and the host's relevance in determining disease occurrence and outcome is now increasingly evident and must be accounted for in developing tomorrow's therapies. Stromal cells and immune cells are emerging as potent drivers of invasion and metastasis and these mice are a significant improvement over immunocompromised animals for studies of these processes. The development of the Xenochimaeric mice model is a step toward reconciling these two notions, allowing us to recreate an individual tumor with important components of the environment in which it originated. These studies will enable a more faithful stroma- and/or immune-directed drug development.

### Example 13

### Expansion of Human Cord Blood-Derived HSCs with Tat-Myc and Tat-Bcl-2

Fresh cord blood cells were obtained from samples that were discarded from a local cord blood bank. All human cells were de-identified and exempt from IRB oversight. Cord blood included O+, O−, A+, A−, B+, B−, and AB+ all of which showed approximately the same expansion profiles.

The total cord volume was split into 20 ml aliquots and diluted 1:1 in PBS. Diluted cord blood (20 mls) was gently overlaid on 20 mls of Ficoll-Paque Plus (Amersham Biosciences Cat #17-1440-03). The cells were spun at 900× gravity for 60 min The buffy coat was removed with a glass pipette and was washed twice with PBS. The cells were resuspended in FCB media (Iscove's (Gibco) supplemented with 10% human plasma, 100 units per ml Penn/Strep, 30 ml of media containing SCF, IL3 and IL6 and 30 mls of media containing TPO, FLT3-L, and GM-CSF described above. FCB media was further supplemented with 5 μg/ml recombinant Tat-Myc, and 10 μg/ml recombinant Tat-Bcl-2 just prior to addition to the fetal cord blood (FCB) cells. The medium was replaced every 3 days over the course of the expansion.

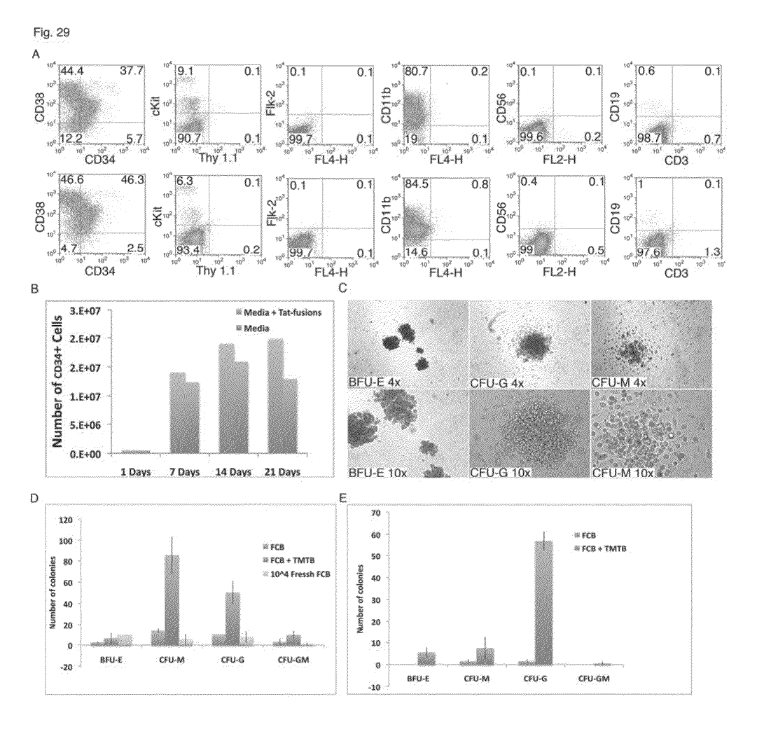

The cytokine cocktail contained IL3, IL6, TPO, Flt3-L, SCF, and GM-CSF which differs from previously reported media in the combination of these six cytokines (Suzuki, T., et al. (2006). Stem Cells 24, 2456-65), as well as by the addition of recombinant Tat-Myc and Tat-Bcl-2. Evaluation of the surface phenotype of the in vitro expanded human HSCs showed that the human HSCs retain their surface characteristics after extended culture in the presence of Tat-Myc and Tat-Bcl-2 (FIG. 29A). This set of conditions resulted in 86.4 fold increase in the number of CD34+ cells in 14 days of culture, and 103.8 fold increase in the number of human CD34+ cells derived from unfractionated cord blood in 21 days of culture (FIG. 29B).

### Example 14

### Tat-Myc and Tat-Bcl-2 Expanded Human CB HSCs are Biologically Active in Vitro and In Vivo

The in vitro expanded human HSCs were plated on MethoCult Optimum (StemCell Technologies), and were examined for their ability to give rise to specific colony types. The in vitro expanded human HSCs are able to give rise to CFU-G, CFU-M, CFU-GM and BFU-E colonies (FIGS. 29C and 29D). In addition, while the surface phenotype of the HSCs expanded in the presence of Tat-Myc and Tat-Bcl-2 was preserved in culture, their colony-forming unit content was significantly enriched under these conditions (FIG. 29D). The CD34+ cells expanded in the presence of Tat-Myc and Tat-Bcl-2 were also able to give rise to new BFU-E, CFU-M, CFU-G and CFU-GM colonies, whereas the CD34+ cells cultured in media alone did not generate new colonies (FIG. 29E).

NOD/SCID/gc−/− mice (NSG) mice were used as recipients for experiments to test the ability of the human CD34+ cells expanded in vitro to give rise to mature human hematopoietic lineages in vivo. This is a documented mouse model useful for this purpose (Tanaka, S., et al. (2012). Development of mature and functional human myeloid subsets in hematopoietic stem cell-engrafted NOD/SCID/IL2rgKO mice. J Immunol 188, 614S—SS).

Fetal cord blood cells (FCBs) were injected into NOD/SCID/gc−/− mice (NSG) mice (Jackson Laboratory) that received 180 rads of radiation just prior to injection. Expanded FCBs were washed 3 times in PBS and injected via the tail vein in 200ul PBS. Eight weeks post-transplant, the mice were bled via the tail vein to assess reconstitution by flow cytometry using the following antibodies: anti-human CD3 (hCD3) (Biolegend Cat #300312), anti-human CD19 (hCD19) (Biolegend Cat #302208) and anti-human CD4S (hCD4S) (Biolegend Cat #304028).

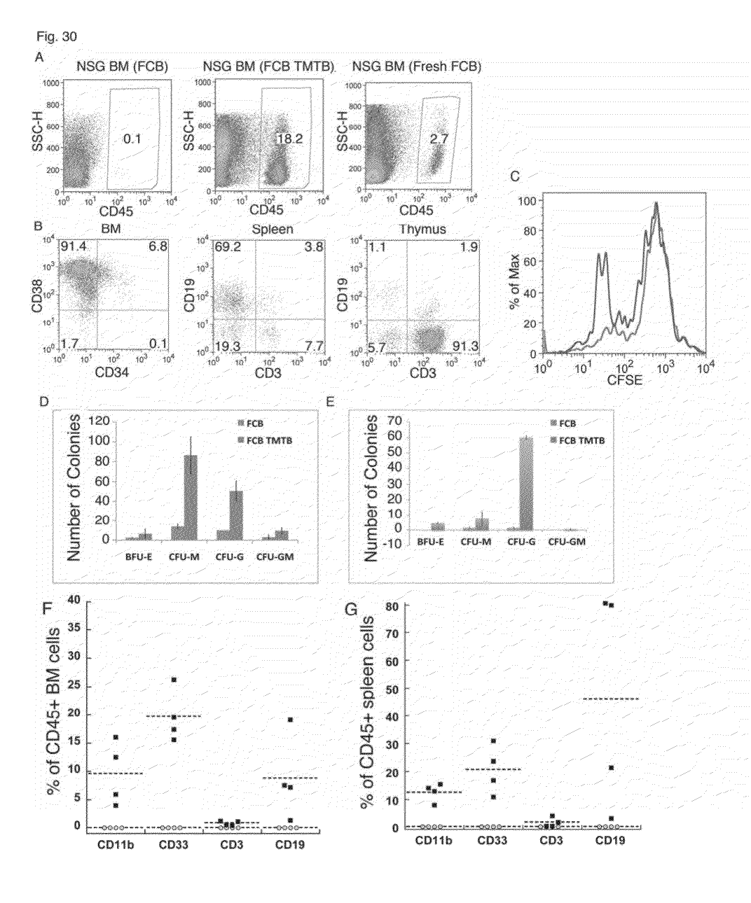

Short term development of human CD4S+ expressing T and B cells in NSG chimaeric mice generated with 1×10⁷ unfractionated cord blood cells was observed. However, the introduction of 1×1 06 protein-transduced long-term (ptlt)-HSC generated in vitro by culture with Tat-Myc and Tat-Bcl-2 for 14 days resulted in a higher frequency of human CD4S+ cells in xenochimaeric NSG mice. In addition, human CD4S+ cells could be observed in the peripheral blood of the mice for up to 20 weeks post transplant (FIG. 30A). Human CD45+, CD34+CD38lo.

HSCs were found in the bone marrow (FIG. 30B), human CD45+/CD3+ and human CD45+/CD19+ lymphoid cells were found in the spleen, and human CD45+, CD3+ lymphoid cells were found in the thymus of xenochimaeric mice.

Human CD45+CD 19+ cells from the spleens of xenochimaeric NSG mice were labeled with CFSE, and were activated with monoclonal antibodies to human CD40 and IgM. The cells were analyzed at 72 hours by flow cytometry for dilution of CFSE. FIG. 30C shows the proliferation profile of the human B-cells that developed in vivo in xenochimaeric NSG mice.

Human CD45+, CD34+CD3810 HSCs from the bone marrow of xenochimaeric NSG mice were used to seed in MethoCult Optimum. These cells gave rise to colonies in MethoCult plates (FIG. 30D), and some of the colonies could still be observed following serial replating (FIG. 30E). The number of colonies in both instances was significantly higher for NSG mice reconstituted with human cord blood cells cultured for 14 days with Tat-Myc and Tat-Bcl-2 than for cells obtained from NSG mice reconstituted with fresh, un-manipulated human cord blood cells.

In addition, a cohort of xenochimaeric mice, engrafted with 10⁶ cord blood cells previously expanded in vitro in a cocktail of cytokines supplemented with Tat-Myc and Tat-Bcl-2 (black squares), were assessed for myeloid and lymphoid cell differentiation. The CD45 positive population of bone marrow cells (FIG. 30F) and spleen cells (FIG. 30G) were analyzed for CD llb, CD33, CD3, and CD19 expression. Both myeloid and lymphoid cell differentiation was observed in the bone marrow and spleen of these xenochimaeric mice.

### Example 15

### Expansion of Human G-CSF Mobilized Peripheral Blood HSCs with Tat-Myc and Tat-Bcl-2

G-CSF mobilized cells were received in a 1 ml volume of elutriated blood from 5 patients who underwent G-CSF mobilization for autologous HSC transplantation. All G-CSF samples were de-identified and no further identifying information was associated with the cells used for these studies. The cells were added drop wise to 10 ml of FCB media. The cells were washed twice in FCB media and treated with 5 μg/ml recombinant Tat-Myc and 10 μg/ml recombinant Tat-Bcl-2 in a 10 ml volume. Cells (5×10⁶) were seeded in the G-Rex 100 cell expansion device (Wilson Wolf Manufacturing) according to the manufacturer's recommendation.

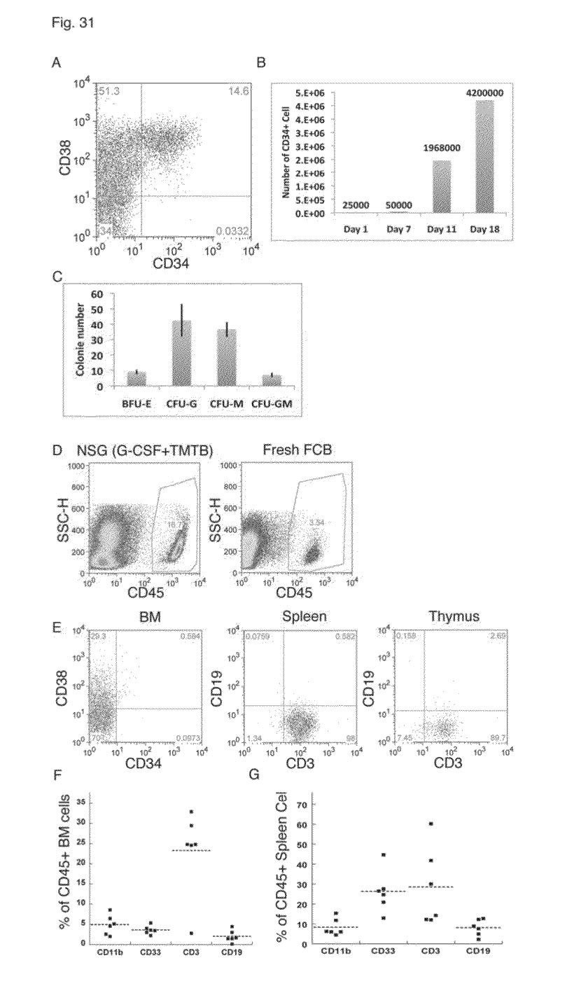

The cells were expanded in media supplemented with cytokines plus Tat-Myc and Tat-Bc12 14 days. The FACS profile of the expanded HSCs shows a distinct population of hCD45+, CD34+, CD38hi, CD133+ cells (FIG. 31A). The kinetics of cell expansion are illustrated in FIG. 31B.

The expanded adult GCS-F mobilized HSCs were then plated on MethoCult Optimum in order to characterize their differentiation potential in vitro. The four colony types normally observed in the media that supports myeloerythroid differentiation were obtained (FIG. 31C), and some of these colony types were also observed upon serial replating.

The expanded adult HSCs were able to reconstitute sublethally irradiated NSG mice. FIG. 31D shows a FACS analysis of the CD45+ staining of bone marrow from NSG mice transplanted 12 weeks earlier with either 10⁶ expanded G-CSF and Tat-Myc/Tat-Bcl-2 mobilized HSCs (first panel) or 5×10⁶ fresh un-manipulated cord blood cells (second panel).

The NSG xenochimaeric mice generated with G-CSF mobilized cells cultured with Tat-Myc and Tat-Bcl-2 were euthanized, and bone marrow, spleen and thymus were collected for further analysis. The analysis of lymphoid organs from xenochimaeric NSG mice reconstituted with expanded adult HSCs showed that there were human CD45+, CD34+CD3810 cells in the bone marrow (FIG. 31E, first panel), human CD45+, CD3+ lymphoid cells in the spleen (FIG. 31E, second panel) and thymus (FIG. 31E, third panel) of those mice. Together, these data demonstrate that one can successfully expand the HSC population obtained from human G-CSF mobilized adult blood.

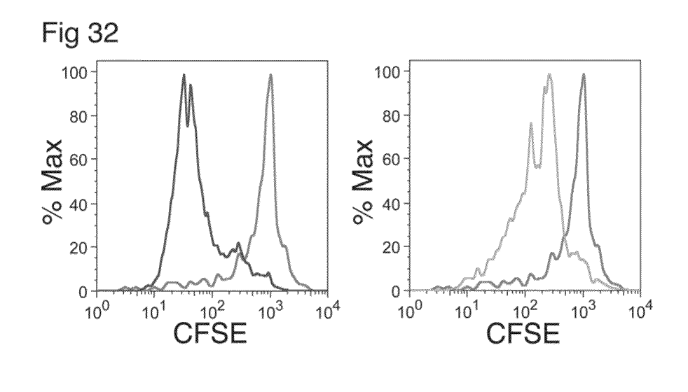

A cohort of xenochimaeric mice engrafted with 10⁶ expanded G-CSF mobilized cells expanded in vitro in a cocktail of cytokines supplemented with Tat-Myc and Tat-Bcl-2 (black squares) were assessed for myeloid and lymphoid cell differentiation. The CD45 positive population of bone marrow cells (FIG. 31F) and spleen cells (FIG. 31G) were analyzed for CD I 1b, CD33, CD3, and CD19 expression. Both myeloid and lymphoid cell differentiation was observed in the bone marrow and spleen of these xenochimaeric mice. In addition, the mature human B-cells derived from the primary xenotranplant responded to stimulation of the antigen receptors in vitro, as determined by CFSE dilution by flow cytometry (FIG. 30C). Similar observations were derived when mature human B cells that developed from the first serial transplant were activated in vitro with antibodies to IgM and CD40 (FIG. 32).

This method is able to generate a sufficient number of HSCs needed for transplantation of an average size adult according to current approaches (Sideri, A, et al. (2011). An overview of the progress on double umbilical cord blood transplantation. Hematologica 96, 1213-20).

1. A method for producing a xenochimaeric mouse, the method comprising
a) introducing heterologous hematopoietic stem cells (HSCs) from a heterologous animal to the mouse, and
b) introducing a solid tumor from the heterologous animal to the mouse.

2. The method of claim 1, wherein the heterologous animal is a human.

3. The method of claim 1, wherein the HSCs and the tumor are from the same individual.

4-11. (canceled)

12. The method of claim 1, wherein the heterologous HSCs are conditionally immortalized HSCs.

13. The method of claim 12, wherein the heterologous HSCs have been conditionally immortalized by culturing the HSCs in the presence of a MYC polypeptide and a BCL-2 polypeptide.

14-15. (canceled)

16. The method of claim 13, wherein the MYC polypeptide is a Tat-MYC polypeptide and/or the BCL-2 polypeptide is a Tat-BCL-2 polypeptide.

17. The method of claim 12, wherein the heterologous HSCs have been conditionally immortalized by introducing nucleic acids encoding myc and/or bcl-2 into the cell.

18-19. (canceled)

20. The method of claim 1, wherein the tumor sample is a solid tumor sample selected from a head and neck tumor, a brain tumor, an eye tumor, a thyroid tumor, an adrenal tumor, a salivary gland tumor, an esophageal tumor, a gastric tumor, an intestinal tumor, a colon tumor, a lung tumor, a breast tumor, a liver tumor, a pancreas tumor, a kidney tumor, a bladder tumor, a prostate tumor, a muscular tumor, an osseous tumor, a skin tumor, and a stroma/sarcoma tumor.

21. A method for producing a population of xenochimaeric mice, the method comprising
a) introducing heterologous HSCs to each individual in a population of the mice, and
b) introducing a portion of a malignant solid tumor from the heterologous animal to each individual in the population of the mice.

22-40. (canceled)

41. A method for evaluating a test agent for treating cancer, the method comprising
a) administering the test agent to one or more xenochimaeric mice produced by the method of claim 1,
b) evaluating the response of the tumor to the test agent.

42-48. (canceled)

49. A method for evaluating the stroma of a tumor sample, the method comprising
a) removing the tumor from a xenochimaeric mouse prepared by the method of claim 1, and
b) detecting the presence of stroma from the heterologous animal in the tumor.

50-59. (canceled)

60. A method of treating a cancer patient, the method comprising administering an effective amount of an anticancer agent to the cancer patient, wherein the anticancer agent was shown to be effective in delaying or inhibiting the growth of a tumor in one or more xenochimaeric mice prepared with HSCs and a tumor sample from the patient according to the methods of claim 1.

61-84. (canceled)

85. The method of claim 3, wherein the individual is a cancer patient.

86. The method of claim 1, wherein the HSCs are introduced to the mice about 8 to about 10 weeks prior to introduction of the tumor to the mice.

87. The method of claim 1, wherein the introduction of the HSCs and the tumor result in formation of stroma corresponding to the heterologous animal.

88. The method of claim 1, wherein the introduction of the HSCs and the tumor result in reversion in one or more of tumor phenotype or tumor genotoype towards the phenotype or genotype of the tumor initially isolated from the heterologous animal.

89. The method of claim 17, wherein the nucleic acid molecule encoding myc comprises MYC-ER.

90. The method of claim 41, wherein the test agent is administered to the xenochimaeric mice about two to about four weeks following introduction of the tumor to the mice.

91. The method of claim 41, wherein the test agent is administered to the xenochimaeric mice after one or more of formation of heterologous stroma, or reversion towards one or more of tumor phenotype or tumor genotoype.

92. A xenochimaeric mouse, comprising:
a) conditionally immortalized heterologous hematopoietic stem cells (HSCs) from a heterologous animal, and
b) a solid tumor from the heterologous animal.
